(19) United States
(12) Patent Application Publication
(10) Pub. No.: US 2022/0009909 A1
(43) Pub. Date: Jan. 13, 2022

(54) ADAMTS INHIBITORS, PREPARATION METHODS AND MEDICINAL USES THEREOF

(71) Applicant: Eternity Bioscience Inc., Cranbury, NJ (US)

(72) Inventors: Dong Liu, Basking Ridge, NJ (US); Peng Zhao, Jamesburg, NJ (US); Jian Liu, Edison, NJ (US); Linghang Zhuang, Chalfont, PA (US); Xinzhu Zhang, Franklin Park, NJ (US); Chunying Song, Newtown, PA (US); Suxing Liu, Edison, NJ (US)

(21) Appl. No.: 17/364,039

(22) Filed: Jun. 30, 2021

Related U.S. Application Data
(60) Provisional application No. 63/175,534, filed on Apr. 15, 2021.
(60) Provisional application No. 63/046,267, filed on Jun. 30, 2020.

(51) Int. Cl. C07D 403/12; A61P 19/02

(52) U.S. Cl. CPC C07D 403/12; C07B 2200/05; A61P 19/02

(57) ABSTRACT

Compounds of formula (I) useful as inhibitors of ADAMTS-5 and/or ADAMTS-4, pharmaceutical compositions thereof, and use of them as therapeutic agents for the treatment of diseases involving degradation of cartilage or disruption of cartilage homeostasis, in particular osteoarthritis and/or rheumatoid arthritis, are disclosed.

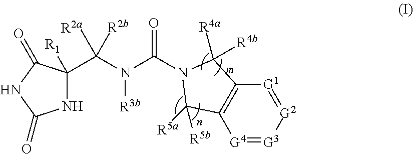

### CROSS-REFERENCE TO RELATED APPLICATIONS

This application claims priority under 35 U.S.C. § 119(e) to U.S. Provisional Patent Application No. 63/046,267, filed on Jun. 30, 2020, and Application. No. 63/175,534, filed on Apr. 15, 2021, the disclosures of both of which are incorporated herein by reference in their entirety.

### FIELD OF THE DISCLOSURE

The present disclosure relates to compounds and methods useful in the inhibition of the function of ADAMTS-5 and/or ADAMTS-4 and accordingly may have a beneficial impact on the therapies of diseases involving degradation of cartilage or disruption of cartilage homeostasis, in particular osteoarthritis and/or rheumatoid arthritis.

### BACKGROUND OF THE DISCLOSURE

Cartilage is the highly specialized connective tissue of diarthrodial joints. Its principal function is to provide the joints the capability of load bearing and compression resistance. Chondrocyte is the cellular component of articular cartilage, taking about only 5% of the tissue volume. The main component of cartilage is extracellular matrix comprising aggrecan and collagen. Under physiological conditions, cartilage homeostasis is maintained by a balance between production (anabolism) and degradation (catabolism) of aggrecan and collagen. However, the balance is shifted to catabolism in diseases such as osteoarthritis.

Osteoarthritis is the most common chronic joint disease and a leading cause of pain and disability in developed countries. It can happen to the joints of the hips, knees, spines, hands and others. It was estimated that 250 million people worldwide are currently being affected by osteoarthritis, and the prevalence is progressively rising (Hunter et al., Lancet. 2019, 393: 1745-1759). Pain and loss of functional capacity are accompanied by an increased risk of additional disease conditions such as diabetes, cancer or cardiovascular disease (Valdes A M and Stocks J. Osteoarthritis and ageing. Eur Med J 2018, 3: 116-123). Osteoarthritis is a whole joint disease, which will lead to degradation of articular cartilage, synovitis and alterations in subchondral bone and other periarticular tissues (Goldring M B and Otero M Inflammation in osteoarthritis. Curr Opin Rheumatol 2011, 23: 471-478). Although the pathogenesis of osteoarthritis is not well understood yet, it is known that osteoarthritis is involved with mechanical damage, inflammation, aging, and metabolism factors. Osteoarthritis is not a passive degenerative disease. In contrast, it is an active dynamic alteration arising from an imbalance between the repair and destruction of joint tissues (Hunter et al., Lancet. 2019, 393: 1745-1759). Currently, the pharmacological treatments available for osteoarthritis are limited to symptomatic relief of pain and inflammation. Disease-modifying drugs that arrest or slow down disease progression are not yet available.

Progressive loss of articular cartilage is currently viewed as an early event in osteoarthritis. Aggrecan may have a role protecting loss of collagen (Pratta et al., J Biol Chem. 2003, 278: 45539-45545). These studies suggest the critical role of aggrecan in osteoarthritis and other joint diseases. Aggrecan is a proteoglycan, possessing a core protein with covalently attached sulfated glycosaminoglycan (GAG) chains. Its core protein has three globular domains, G1 and G2 domains in the N-terminus, and G3 in the C-terminus. The extensive region between the G2 and G3 domains is heavily modified by (GAG) keratan sulfate (KS) and chondroitin sulfate (CS). Based on the differences in the amino acid sequence, the CS domain is further divided into two subdomains, CS1 and CS2. The GAG chains provide aggrecan with its high anionic charge. Multiple aggrecan monomers bind to hyaluronan (HA) through G1 domains, which is stabilized by a link protein, forming large supramolecular aggregates. The large-sized aggrecan aggregates absorb water and provide the resilient properties for the cartilage (Roughley et al., The Journal of Experimental Orthopaedics. 2014, 1: 8). A high concentration of aggrecan, a high degree of sulfation and the ability to form large aggregation are required for the normal function of cartilage.

The extended structure of aggrecan can be cleaved by proteolytic enzymes, leading to impaired normal function of cartilage. ADAMTS (a disintegrin and metalloproteinase with thrombospondin motifs) is a family of zinc ion-dependent metalloproteases. ADAMTS-4 and -5, also termed “aggrecanases”, degrade aggrecan at several specific locations in the IGD and the CS2 domains. It was demonstrated that ADAMTS-5 deficiency protects against aggrecan loss and cartilage damage in mouse osteoarthritis disease model induced by surgeries (Glasson et al., Nature, 2005, 434: 644-648; Stanton et al., Nature. 2005, 434: 648-652), implicating ADAMTS-5 in driving cartilage loss and osteoarthritis disease severity. However, some studies in human cartilage explant culture suggested that not only ADAMTS-5 but also ADAMTS-4 is important for human osteoarthritis (Verina et al., Journal of Cellular Biochemistry. 2011, 112: 3507-3514). These studies strongly suggest that inhibiting the enzymatic function of ADAMTS-5 and ADAMTS-4 may provide a protecting role in osteoarthritis.

In summary, the role of ADAMTS-5 and/or ADAMTS-4 in cartilage degradation has been well-established. Therefore, compounds that can inhibit ADAMTS-5 and/or ADAMTS-4 may be of therapeutic value in the treatment of arthritis.

Patent applications for compounds that have been published as ADAMTS-5 and/or ADAMTS-4 inhibitors include WO2014066151A1, WO2016102347A1, WO2017211667A1, WO2017211666A1, WO2017211668A1, WO2021011720A2 and WO2021011723A1.

### SUMMARY OF THE DISCLOSURE

The compounds of this disclosure inhibit the function of ADAMTS-5 and/or ADAMTS-4 and accordingly may serve as therapeutic agents for the treatment of diseases involving degradation of cartilage or disruption of cartilage homeostasis, in particular, osteoarthritis and/or rheumatoid arthritis.

The present disclosure, in one aspect, provides a compound of formula (I), or a tautomer, mesomer, racemate, enantiomer, diastereomer thereof, or mixture thereof, or a pharmaceutically acceptable salt, solvate or prodrug thereof:

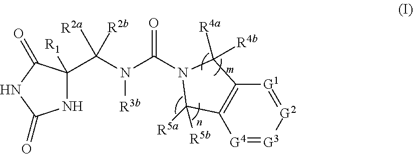

wherein:

G¹, G², G³ and G⁴ are each independently N or CR⁶, provided that no more than two of them are N;

R¹ is selected from the group consisting of hydrogen, alkyl, haloalkyl, hydroxyalkyl, cycloalkyl, heterocyclyl and heteroaryl, wherein the alkyl, cycloalkyl, heterocyclyl and heteroaryl are each optionally substituted with one or more, preferably one to five, and sometimes more preferably one to three, groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NH C(═O)R^{11a}, NH C(═O)OR^{11a}, cycloalkyl, heterocyclyl, aryl and heteroaryl;

R^{2a} and R^{2b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;

R^{3b} is selected from the group consisting of hydrogen, alkyl, hydroxy, haloalkyl, hydroxyalkyl, amino, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;

or two of R^{2a}, R^{2b} and R^{3b} together form cycloalkyl or heterocyclyl;

R^{4a}, R^{4b}, R^{5a} and R^{5b} are each identical or different, and each is independently selected from the group consisting of hydrogen, deuterium, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is each optionally substituted with one or more, preferably one to five, and sometimes more preferably one to three, groups independently selected from the group consisting of halogen, alkyl, alkoxy, hydroxy, hydroxyalkyl, cyano, amino, nitro, cycloalkyl, heterocyclyl, aryl and heteroaryl; or alternatively two of R^{4a}, R^{4b}, R^{5a} and R^{5b} together can form cycloalkyl or heterocyclyl;

R⁶ at each occurrence is identical or different, and each is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}), C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl or heteroaryl is optionally substituted with one or more, preferably one to five, and sometimes more preferably one to three, groups selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;

each of R^{11a}, R^{12a}, R^{13a}, and R^{14a} is independently selected from the group consisting of hydrogen, alkyl, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, hydroxy, alkoxy, alkyl, and cycloalkyl;

each of R^{11b}, R^{12b}, R^{13b}, and R^{14b} is independently selected from the group consisting of hydrogen and alkyl, wherein alkyl is optionally substituted with one or more, preferably one to three, groups independently selected from the group consisting of halogen, hydroxyl and alkoxy;

n is 1 or 2; and

m is 1 or 2.

In another aspect, this disclosure provides a preparation process of a compound of formula (I), or a tautomer, mesomer, racemate, enantiomer, diastereomer, or mixture thereof, or a pharmaceutically acceptable salt thereof, solvate or prodrug thereof, the preparation process comprising the steps of:

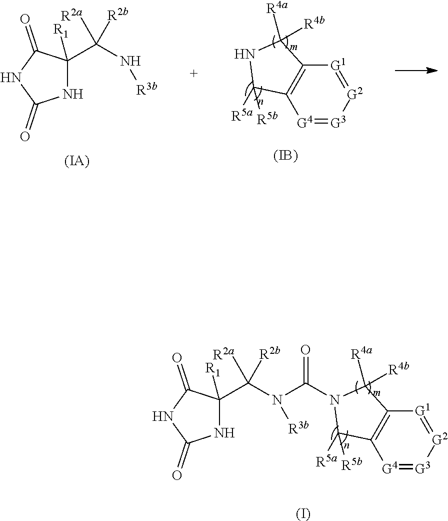

reacting a compound of formula (IA) with an acylation reagent to generate a reactive carbamate intermediate which can then be reacted with a compound of formula (IB) to obtain the compound of formula (I), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride;

the acylation reagent is preferably 4-nitrophenyl chloroformate; and

G¹, G², G³, G⁴, R¹, R^{2a}, R^{4a} to R^{5a}, R^{2b} to R^{5b}, n and m are each as defined in formula (I).

In another aspect, the present disclosure provides a pharmaceutical composition comprising a compound of formula (I), or a tautomer, mesomer, racemate, enantiomer, diastereomer, or mixture thereof, or a pharmaceutically acceptable salt, solvate or prodrug thereof, and a pharmaceutically acceptable carrier.

In another aspect, the present disclosure provides a method of inhibiting ADAMTS-5 and/or ADAMTS-4, comprising administering to a subject in need thereof a therapeutically effective amount of a compound of formula (I), or a tautomer, mesomer, racemate, enantiomer, diastereomer, or mixture thereof, or a pharmaceutically acceptable salt, solvate or prodrug thereof, or the pharmaceutical composition containing the compound.

In another aspect, the present disclosure also provides a method of preventing and/or treating an inflammatory condition, a disease involving degradation of cartilage and/or disruption of cartilage homeostasis, comprising administering to a subject in need thereof a therapeutically effective amount of a compound of formula (I), or a tautomer, mesomer, racemate, enantiomer, diastereomer, or mixture thereof, or a pharmaceutically acceptable salt, solvate or prodrug thereof, or the pharmaceutical composition containing the compound.

Other aspects or advantages of the disclosure will be better appreciated in view of the following detailed description, examples, and claims.

### DETAILED DESCRIPTION OF THE DISCLOSURE

In one aspect, the present disclosure provides a compound of formula (I), or a tautomer, mesomer, racemate, enantiomer, diastereomer thereof, or mixture thereof, or a pharmaceutically acceptable salt, solvate or prodrug thereof:

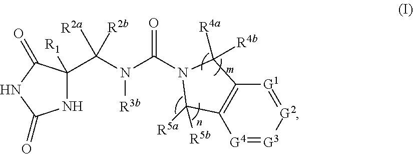

wherein G¹, G², G³, G⁴, R¹, R^{2a}, R^{4a} to R^{5a}, R^{2b} to R^{5b}, n and m are each as defined above.

In some embodiments of the disclosure, the compound of formula (I), or a tautomer, mesomer, racemate, enantiomer, diastereomer, or mixture thereof, or a pharmaceutically acceptable salt thereof, solvate or prodrug thereof, is a compound of formula. (II), or a tautomer, mesomer, racemate, enantiomer, diastereomer, or mixture thereof, or a pharmaceutically acceptable salt thereof, solvate or prodrug thereof:

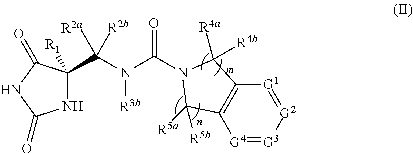

wherein G¹, G², G³, G⁴, R¹, R^{2a}, R^{4a} to R^{5a}, R^{2b} to R^{5b}, n and m are each as defined in formula (I).

In some embodiments of the disclosure, the compound of formula (I), or a tautomer, mesomer, racemate, enantiomer, diastereomer, or mixture thereof, or a pharmaceutically acceptable salt thereof, solvate or prodrug thereof, is a compound of formula (IIa), or a tautomer, mesomer, racemate, enantiomer, diastereomer, or mixture thereof, or a pharmaceutically acceptable salt thereof, solvate or prodrug thereof:

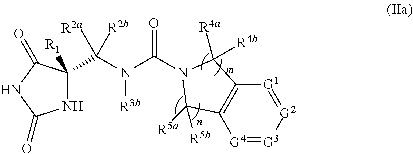

wherein G¹, G², G³, G⁴, R¹, R^{2a}, R^{4a} to R^{5a}, R^{2b} to R^{5b}, n and in are each as defined in formula (I).

In some embodiments of the disclosure, in the compound of formula (I), (II) or (IIa), or a tautomer, mesomer, racemate, enantiomer, diastereomer, or mixture thereof, or a pharmaceutically acceptable salt thereof, solvate or prodrug thereof, G¹ and G² are each independently N or CR⁶; G³ and G⁴ are each CR⁶; and R⁶ is as defined in formula (I), (II) or (IIa).

In some embodiments of the disclosure, the compound of formula (I) or (II), or a tautomer, mesomer, racemate, enantiomer, diastereomer, or mixture thereof, or a pharmaceutically acceptable salt thereof, solvate or prodrug thereof, is a compound of formula (III) or (IIIa), or a tautomer, mesomer, racemate, enantiomer, diastereomer, or mixture thereof, or a pharmaceutically acceptable salt thereof, solvate or prodrug thereof:

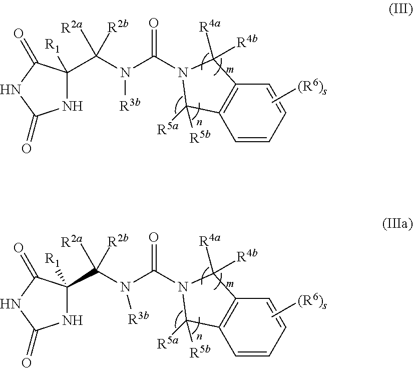

wherein s is 0, 1, 2, 3 or 4; and

R¹, R^{2a}, R^{4a} to R^{5a}, R^{2b} to R^{5b}, R⁶, n and m are each as defined in formula (I) or (II).

In some embodiments of the disclosure, in the compound of formula (I), (II), (IIa), (III) or (IIIa), or a tautomer, mesomer, racemate, enantiomer, diastereomer, or mixture thereof, or a pharmaceutically acceptable salt thereof, solvate or prodrug thereof, R¹ is cycloalkyl; preferably is 3-6 membered cycloalkyl; more preferably is cyclopropyl.

In some embodiments of the disclosure, in the compound of formula (I), (II), (IIa), (III) or (IIIa), or a tautomer, mesomer, racemate, enantiomer, diastereomer, or mixture thereof, or a pharmaceutically acceptable salt thereof, solvate or prodrug thereof, R^{2a} and R^{2b} are each independently selected from the group consisting of hydrogen, halogen, alkyl, alkoxy, haloalkyl, haloalkoxy, hydroxyalkyl and cycloalkyl, wherein the alkyl or cycloalkyl is optionally substituted with one or more, preferably one to five, and sometimes more preferably one to three, groups selected from the group consisting of halogen, alkyl, alkoxy, hydroxy, hydroxyalkyl and cycloalkyl; and sometimes preferably R^{2a} and R^{2b} are each independently selected from the group consisting of hydrogen, halogen, C₁₋₆ alkyl and C₁₋₆ haloalkyl; more preferably R^{2a} and R^{2b} are each hydrogen.

In some embodiments of the disclosure, the compound of formula (I), (II) or (III), or a tautomer, mesomer, racemate, enantiomer, diastereomer, or mixture thereof, or a pharmaceutically acceptable salt thereof, solvate or prodrug thereof, is a compound of formula (IV) or (IVa), or a tautomer, mesomer, racemate, enantiomer, diastereomer, or mixture thereof, or a pharmaceutically acceptable salt thereof, solvate or prodrug thereof:

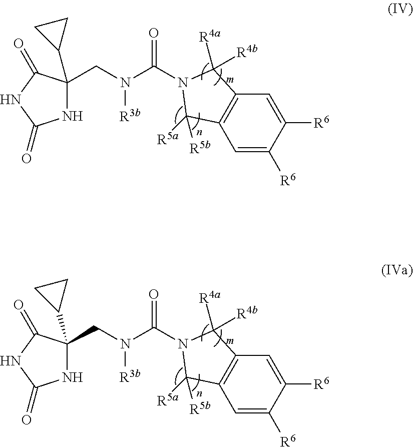

wherein R^{4a}, R^{5a}, R^{3b} to R^{5b}, R⁶, n and m are each as defined in formula (I), (II) or (III).

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, R^{3b} is selected from the group consisting of hydrogen, alkyl and hydroxy alkyl, wherein the alkyl is optionally substituted with one or more, one or more, preferably one to three, groups independently selected from the group consisting of halogen and alkoxy.

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, R^{3b} is selected from the group consisting of hydrogen and alkyl; preferably R^{3b} is hydrogen or C₁₋₆ alkyl; more preferably is hydrogen or methyl.

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, R^{4a}, R^{4b}, R^{5a} and R^{5b} are each identical or different, and each is independently selected from the group consisting of hydrogen, deuterium, halogen and alkyl.

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, R^{4a}, R^{4b}, R^{5a} and R^{5b} are each identical or different, and each is independently selected from the group consisting of hydrogen, deuterium and alkyl; preferably R^{4a}, R^{4b}, R^{5a} and R^{5b} are each identical or different, and each is independently selected from the group consisting of hydrogen, deuterium and C₁₋₆ alkyl; more preferably R^{4a}, R^{4b}, R^{5a} and R^{5b} are each identical or different, and each is independently selected from the group consisting of hydrogen, deuterium and methyl.

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each R⁶ is identical or different, and at each occurrence is independently selected from the group consisting of hydrogen, halogen, alkyl, alkoxy, haloalkyl, haloalkoxy and cyano.

In some embodiments of the disclosure, in the compound of formula (I), (II), (II a), (III), (IIIa), (IV) or (IVa), or a tautomer, mesomer, racemate, enantiomer, diastereomer, or mixture thereof, or a pharmaceutically acceptable salt thereof, solvate or prodrug thereof, each R⁶ is identical or different, and at each occurrence is independently selected from the group consisting of hydrogen, halogen and haloalkyl; preferably R⁶ is identical or different, and at each occurrence is independently selected from the group consisting of hydrogen, halogen and C₁₋₆ haloalkyl; more preferably R⁶ is identical or different, and at each occurrence is independently selected from the group consisting of hydrogen, halogen, CF₃ and CHF₂.

In some embodiments of the disclosure, in the compound of formula (I), (II) or (IIa), or a tautomer, mesomer, racemate, enantiomer, diastereomer, or mixture thereof, or a pharmaceutically acceptable salt thereof, solvate or prodrug thereof,

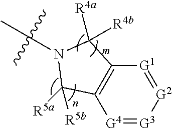

is selected from the group consisting of

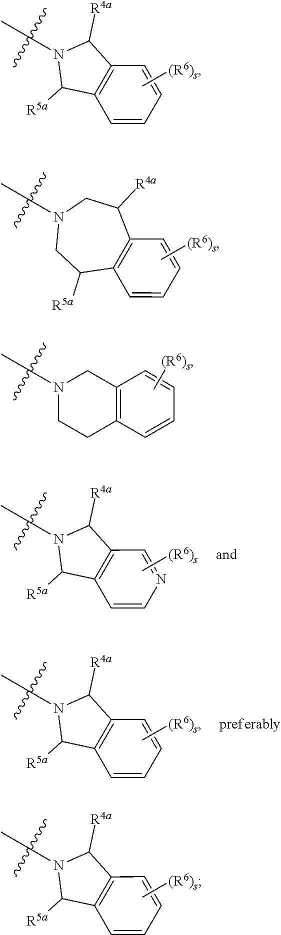

R^{4a}, R^{5a}, and R⁶ are as defined in formula (I), and s is defined in formula (III); preferably s is 1 or 2.

In some embodiments of the disclosure, in the compound of formula (III) or (IIIa), or a tautomer, mesomer, racemate, enantiomer, diastereomer, or mixture thereof, or a pharmaceutically acceptable salt thereof, solvate or prodrug thereof,

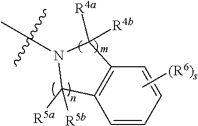

is selected from the group consisting of

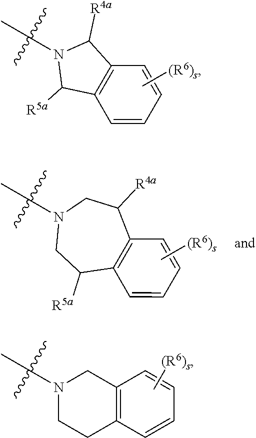

preferably

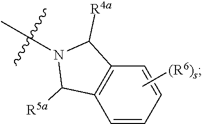

R^{4a}, R^{5a} and R⁶ are as defined in formula (I) and s is defined in formula (III).

In some embodiments of the disclosure, in the compound of formula (IV) or (IVa), or a tautomer, mesomer, racemate, enantiomer, diastereomer, or mixture thereof, or a pharmaceutically acceptable salt thereof, solvate or prodrug thereof,

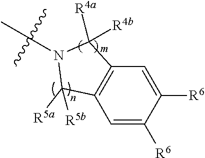

is selected from the group consisting of

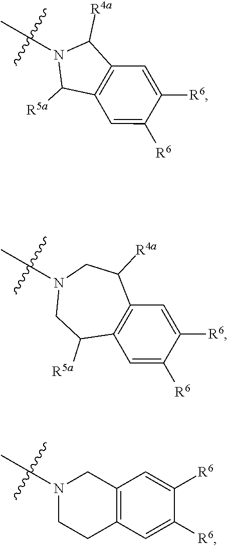

preferably

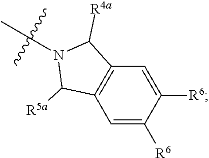

R^{4a}, R^{5a} and R⁶ are as defined in formula (I).

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, m is 1.

In some embodiments of the disclosure, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, n is 1.

In some embodiments of the disclosure, in the compound of formula (III) and formula (IIIa), or a tautomer, mesomer, racemate, enantiomer, diastereomer, or mixture thereof, or a pharmaceutically acceptable salt thereof, solvate or prodrug thereof, s is 1 or 2.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 52.5%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof each designated deuterium atom has deuterium incorporation of at least 60%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 67.5%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 75%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 82.5%.

In some embodiments, in the compound of formula (I), (II), (IIa), (Iii), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 90%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 95%.

In some embodiments, in the compound of formula (I), (II), (IIa), (III), (IIIa), (IV) or

(IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 97%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 97.5%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 98%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 99%.

In some embodiments, in the compound of formula (I), (II), (IIa), (III), (IIIa), (IV) or (IVa), or a tautomer, mesomer, racemate, enantiomer, diastereomer, or mixture thereof, or a pharmaceutically acceptable salt thereof, solvate or prodrug thereof, each designated deuterium atom has deuterium incorporation of at least 99.5%.

In some embodiments of the disclosure, in the compound of formula (II) or (IIa), or a tautomer, mesomer, racemate, enantiomer, diastereomer, or mixture thereof, or a pharmaceutically acceptable salt thereof, solvate or prodrug thereof, wherein G¹ and G² are each independently N or CR⁶; G³ and G⁴ are each CR⁶; R⁶ is identical or different, and at each occurrence is independently selected from the group consisting of hydrogen, halogen and C₁₋₆ haloalkyl; R¹ is 3-6 membered cycloalkyl; R^{2a} and R^{2b} are each independently selected from the group consisting of hydrogen, halogen, C₁₋₆ alkyl and C₁₋₆ haloalkyl; R^{3b} is hydrogen or C₁₋₆ alkyl; R^{4a}, R^{4b}, R^{5a} and R^{5b} are each identical or different, and each is independently selected from the group consisting of hydrogen, deuterium and C₁₋₆ alkyl; m is 1; and n is 1.

In some embodiments of the disclosure, in the compound of formula (III) or (IIIa), or a tautomer, mesomer, racemate, enantiomer, diastereomer, or mixture thereof, or a pharmaceutically acceptable salt thereof, solvate or prodrug thereof, wherein R¹ is cyclopropyl; R^{2a} and R^{2b} are each independently selected from the group consisting of hydrogen, halogen, C₁₋₆ alkyl and C₁₋₆ haloalkyl; R^{3b} is hydrogen or C₁₋₆ alkyl; R^{4a}, R^{4b}, R^{5a} and R^{5b} are each identical or different, and each is independently selected from the group consisting of hydrogen, deuterium and C₁₋₆ alkyl; R⁶ is identical or different, and at each occurrence is independently selected from the group consisting of hydrogen, halogen and C₁₋₆ haloalkyl; m is 1; n is 1; and s is 1 or 2.

In some embodiments of the disclosure, in the compound of formula (IV) or (IVa), or a tautomer, mesomer, racemate, enantiomer, diastereomer, or mixture thereof, or a pharmaceutically, acceptable salt thereof, solvate or prodrug thereof, wherein R^{3b} is hydrogen or C₁₋₆ alkyl; R^{4a}, R^{4b}, R^{5a} and R^{5b} are each identical or different, and each is independently selected from the group consisting of hydrogen, deuterium and C₁₋₆ alkyl; R⁶ is identical or different, and at each occurrence is independently selected from the group consisting of hydrogen, halogen and C₁₋₆ haloalkyl; m is 1; and n is 1.

TABLE A
Exemplified compounds of the disclosure include, but are not limited to:

| Example | |
|---|---|
| No. | Compound structure and name |
| 1 | 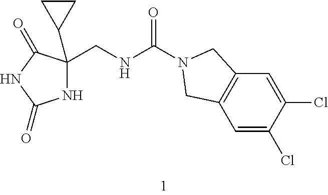 [SMILES: O=C1NC(=O)C(CNC(=O)N2Cc3cc(Cl)c(Cl)cc3C2)(C2CC2)N1] |
| | 5,6-dichloro-N-((4-cyclopropyl-2,5-dioxoimidazolidin-4- |
| | yl)methyl)isoindoline-2-carboxamide 1 |
| 1-1 | 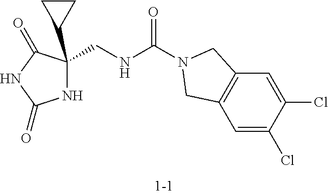 [SMILES: O=C1NC(=O)[C@@](CNC(=O)N2Cc3cc(Cl)c(Cl)cc3C2)(C2CC2)N1] |
| | (S)-5,6-dichloro-N-((4-cyclopropyl-2,5-dioxoimidazolidin-4- |
| | yl)methyl)isoindoline-2-carboxamide 1-1 |
| 1-2 | 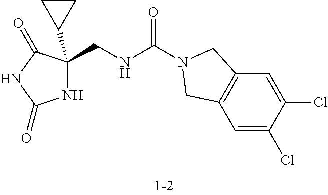 [SMILES: O=C1NC(=O)[C@](CNC(=O)N2Cc3cc(Cl)c(Cl)cc3C2)(C2CC2)N1] |
| | (R)-5,6-dichloro-N-((4-cyclopropyl-2,5-dioxoimidazolidin-4- |
| | yl)methyl)isoindoline-2-carboxamide 1-2 |
| 2 | 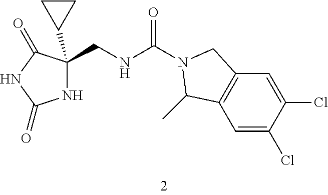 [SMILES: CC1c2cc(Cl)c(Cl)cc2CN1C(=O)NC[C@@]1(C2CC2)NC(=O)NC1=O] |
| | 5,6-dichloro-N-(((R)-4-cyclopropyl-2,5-dioxoimidazolidin-4- |
| | yl)methyl)-1-methylisoindoline-2-carboxamide 2 |
| 2-1 | 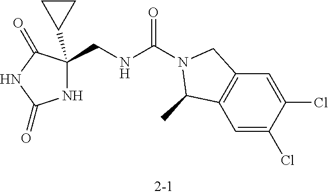 [SMILES: C[C@@H]1c2cc(Cl)c(Cl)cc2CN1C(=O)NC[C@@]1(C2CC2)NC(=O)NC1=O] |
| | (R)-5,6-dichloro-N-(((R)-4-cyclopropyl-2,5-dioxoimidazolidin-4- |
| | yl)methyl)-1-methylisoindoline-2-carboxamide 2-1 |
| 2-2 | 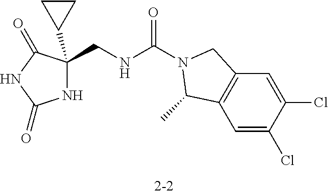 [SMILES: C[C@H]1c2cc(Cl)c(Cl)cc2CN1C(=O)NC[C@@]1(C2CC2)NC(=O)NC1=O] |
| | (R)-5,6-dichloro-N-(((R)-4-cyclopropyl-2,5-dioxoimidazolidin-4- |
| | yl)methyl)-1-methylisoindoline-2-earboxamide 2-2 |
| 3 | 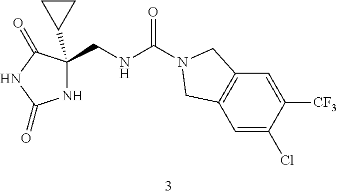 [SMILES: O=C1NC(=O)[C@](CNC(=O)N2Cc3cc(Cl)c(C(F)(F)F)cc3C2)(C2CC2)N1] |
| | (R)-5-chloro-N-((4-cyclopropyl-2,5-dioxoimidazolidin-4-yl) |
| | methyl)-6-(trifluoromethyl)isoindoline-2-carboxamide 3 |
| 4 | 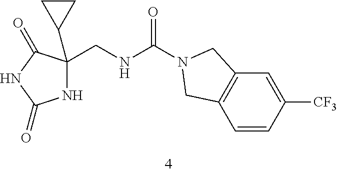 [SMILES: O=C1NC(=O)C(CNC(=O)N2Cc3ccc(C(F)(F)F)cc3C2)(C2CC2)N1] |
| | N-((4-cyclopropyl-2,5-dioxoimidazolidin-4-yl)methyl)-5- |
| | (trifluoromethyl)isoindoline-2-carboxamide 4 |
| 5 | 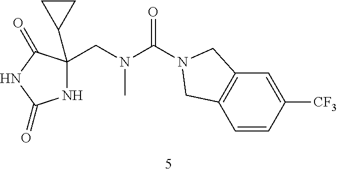 [SMILES: CN(CC1(C2CC2)NC(=O)NC1=O)C(=O)N1Cc2ccc(C(F)(F)F)cc2C1] |
| | N-(((4-cyclopropyl-2,5-dioxoimidazolidin-4-yl)methyl)-N-methyl-5- |
| | (trifluoromethyl)isoindoline-2-earboxamide 5 |
| 8 | 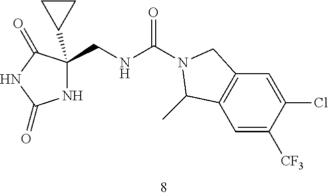 [SMILES: CC1c2cc(C(F)(F)F)c(Cl)cc2CN1C(=O)NC[C@@]1(C2CC2)NC(=O)NC1=O] |
| | 5-chloro-N-(((R)-4-cyclopropyl-2,5-dioxoimidazolidin-4-yl) |
| | methyl)-1-methyl-6-(trifluoromethyl)isoindoline-2-carboxamide |
| 8-1 | 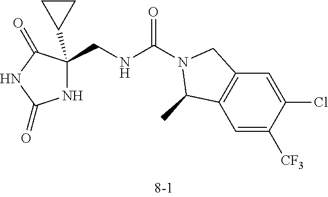 [SMILES: C[C@@H]1c2cc(C(F)(F)F)c(Cl)cc2CN1C(=O)NC[C@@]1(C2CC2)NC(=O)NC1=O] |
| | (R)-5-chloro-N-(((R)-4-cyclopropyl-2,5-dioxoimidazolidin-4- |
| | yl)methyl)-1-methyl-6-(trifluoromethyl)isoindoline-2-carboxamide |
| 8-2 | 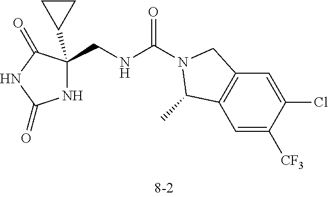 [SMILES: C[C@H]1c2cc(C(F)(F)F)c(Cl)cc2CN1C(=O)NC[C@@]1(C2CC2)NC(=O)NC1=O] |
| | (5)-5-chloro-N-(((R)-4-cyclopropyl-2,5-dioxoimidazolidin-4- |
| | yl)methyl)-1-methyl-6-(trifluoromethyl)isoindoline-2-carboxamide |
| 9 | 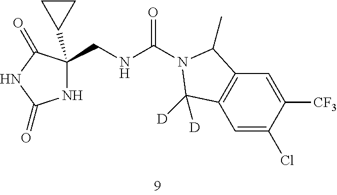 [SMILES: [2H]C1([2H])c2cc(Cl)c(C(F)(F)F)cc2C(C)N1C(=O)NC[C@@]1(C2CC2)NC(=O)NC1=O] |
| | 5-chloro-N-(((R)-4-cyclopropyl-2,5-dioxoimidazolidin-4-yl) |
| | methyl)-1-methyl-6-(trifluoromethyl)isoindoline-3,3-d₂- |
| | 2-carboxamide |
| 11 | 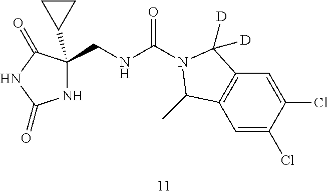 [SMILES: [2H]C1([2H])c2cc(Cl)c(Cl)cc2C(C)N1C(=O)NC[C@@]1(C2CC2)NC(=O)NC1=O] |
| | 5,6-dichloro-N-(((R)-4-cyclopropyl-2,5-dioxoimidazolidin-4- |
| | yl)methyl)-1-methylisoindoline-3,3-d₂-2-carboxamide |
| 12 | 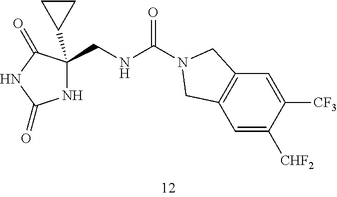 [SMILES: O=C1NC(=O)[C@](CNC(=O)N2Cc3cc(C(F)F)c(C(F)(F)F)cc3C2)(C2CC2)N1] |
| | (R)-N-((4-cyc1opropyl-2,5-dioxoimidazo1idin-4-yl)methyl)-5- |
| | (difluoromethyl)-6-(trifluoromethyl)isoindoline-2-carboxamide |

TABLE B
The following compounds could be prepared:

| | |
|---|---|
| 6 | 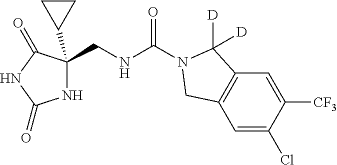 [SMILES: [2H]C1([2H])c2cc(C(F)(F)F)c(Cl)cc2CN1C(=O)NC[C@@]1(C2CC2)NC(=O)NC1=O] |
| 7 | 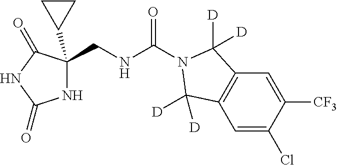 [SMILES: [2H]C1([2H])c2cc(Cl)c(C(F)(F)F)cc2C([2H])([2H])N1C(=O)NC[C@@]1(C2CC2)NC(=O)NC1=O] |
| 10 | 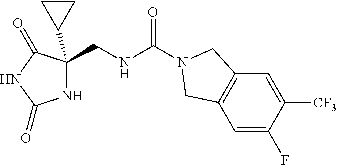 [SMILES: O=C1NC(=O)[C@](CNC(=O)N2Cc3cc(F)c(C(F)(F)F)cc3C2)(C2CC2)N1] |
| 13 | 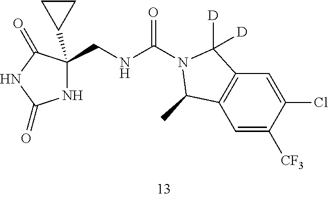 [SMILES: [2H]C1([2H])c2cc(Cl)c(C(F)(F)F)cc2[C@@H](C)N1C(=O)NC[C@@]1(C2CC2)NC(=O)NC1=O] |
| 14 | 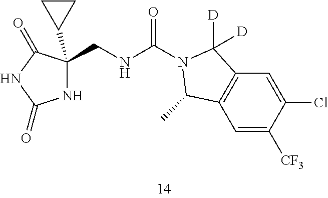 [SMILES: [2H]C1([2H])c2cc(Cl)c(C(F)(F)F)cc2[C@H](C)N1C(=O)NC[C@@]1(C2CC2)NC(=O)NC1=O] |

In another aspect, this disclosure provides a preparation process of a compound of formula (I), or a tautomer, mesomer, racemate, enantiomer, diastereomer, or mixture thereof, or a pharmaceutically acceptable salt thereof, solvate or prodrug thereof, the preparation process comprising the steps of:

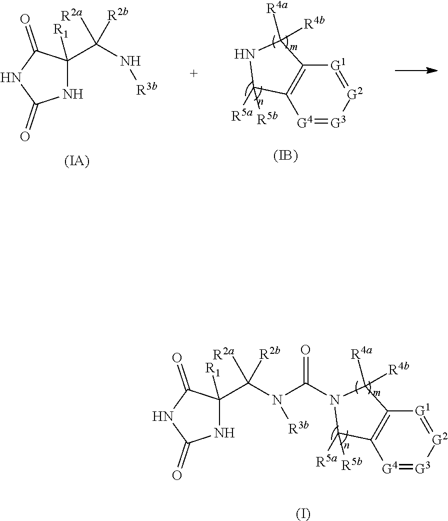

reacting a compound of formula (IA) with an acylation reagent to generate a reactive carbamate intermediate which can then be reacted with a compound of formula (IB) to obtain the compound of formula (I), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride;

the acylation reagent is preferably 4-nitrophenyl chloroformate; and

G¹, G², G³, G⁴, R¹, R^{2a}, R^{4a} to R^{5a}, R^{2b} to R^{5b}, n and m are each as defined in formula (I).

In another aspect, this disclosure provides a preparation process of a compound of formula (II), or a tautomer, mesomer, racemate, enantiomer, diastereomer, or mixture thereof, or a pharmaceutically acceptable salt thereof, solvate or prodrug thereof, the preparation process comprising the steps of:

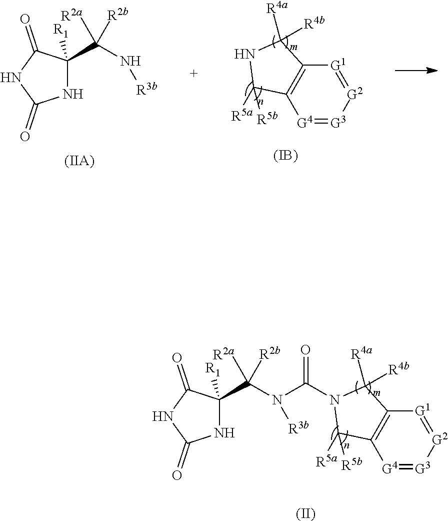

reacting a compound of formula (IIA) with an acylation reagent to generate a reactive carbamate intermediate which can then be reacted with a compound of formula (IB) to obtain the compound of formula (II), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride;

the acylation reagent is preferably 4-nitrophenyl chloroformate; and

G¹, G², G³, G⁴, R¹, R^{2a}, R^{4a} to R^{5a}, R^{2b} to R^{5b}, n and m are each as defined in formula (II).

In another aspect, this disclosure provides a preparation process of a compound of formula (IIa), or a tautomer, mesomer, racemate, enantiomer, diastereomer, or mixture thereof, or a pharmaceutically acceptable salt thereof, solvate or prodrug thereof, the preparation process comprising the steps of:

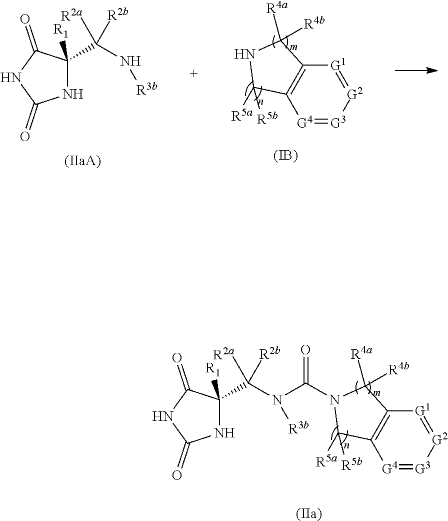

reacting a compound of formula (IIaA) with an acylation reagent to generate a reactive carbamate intermediate which can then be reacted with a compound of formula (IB) to obtain the compound of formula. (IIa), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride;

the acylation reagent is preferably 4-nitrophenyl chloroformate; and

G¹, G², G³, G⁴, R¹, R^{2a}, R^{4a} to R^{5a}, R^{2b} to R^{5b}, n and in are each as defined in formula (IIa).

In another aspect, this disclosure provides a preparation process of a compound of formula (III), or a tautomer, mesomer, racemate, enantiomer, diastereomer, or mixture thereof, or a pharmaceutically acceptable salt thereof, solvate or prodrug thereof, the preparation process comprising the steps of:

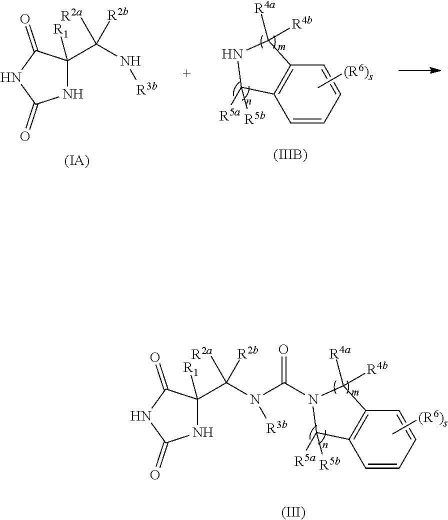

reacting a compound of formula (IA) with an acylation reagent to generate a reactive carbamate intermediate which can then be reacted with a compound of formula (IIIB) to obtain the compound of formula (III), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride, but not limited to;

the acylation reagent is preferably 4-nitrophenyl chloroformate; and

R¹, R^{2a}, R^{4a} to R^{5a}, R^{2b} to R^{5b}, R⁶, n, m and s are each as defined in formula (III).

In another aspect, this disclosure provides a preparation process of a compound of formula (IIIa), or a tautomer, mesomer, racemate, enantiomer, diastereomer, or mixture thereof, or a pharmaceutically acceptable salt thereof, solvate or prodrug thereof, the preparation process comprising the steps of:

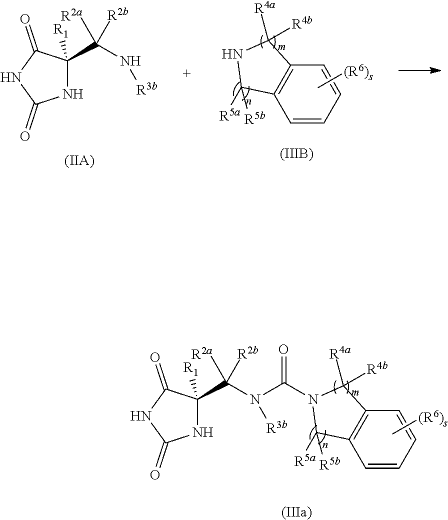

reacting a compound of formula (IIA) with an acylation reagent to generate a reactive carbamate intermediate which can then be reacted with a compound of formula (IIIB) to obtain the compound of formula (IIIa), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride, but not limited to;

the acylation reagent is preferably 4-nitrophenyl chloroformate; and

R¹, R^{2a}, R^{4a} to R^{5a}, R^{2b} to R^{5b}, R⁶, n, m and s are each as defined in formula (IIIa).

In another aspect, this disclosure provides a preparation process of a compound of formula (IV), or a tautomer, mesomer, racemate, enantiomer, diastereomer, or mixture thereof, or a pharmaceutically acceptable salt thereof, solvate or prodrug thereof, the preparation process comprising the steps of:

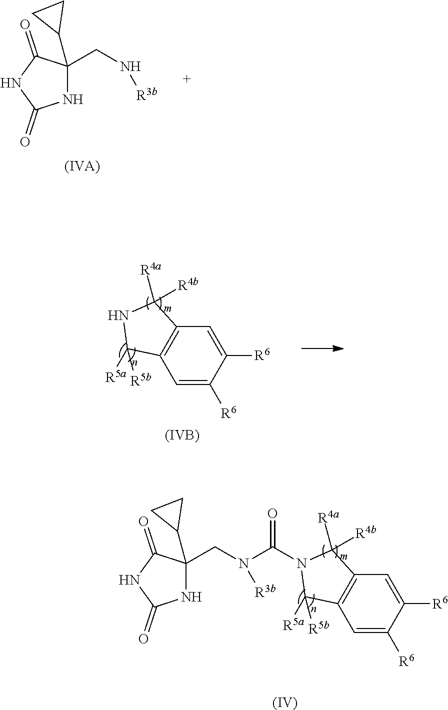

reacting a compound of formula. (IVA) with an acylation reagent to generate a reactive carbamate intermediate which can then be reacted with a compound of formula (IVB) to obtain the compound of formula (IV), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride, but not limited to;

the acylation reagent is preferably 4-nitrophenyl chloroformate; and

R^{4a} to R^{5a}, R^{3b} to R^{5b}, R⁶, m and n are each as defined in formula (IV).

In another aspect, this disclosure provides a preparation process of a compound of formula (IVa), or a tautomer, mesomer, racemate, enantiomer, diastereomer, or mixture thereof, or a pharmaceutically acceptable salt thereof, solvate or prodrug thereof, the preparation process comprising the steps of:

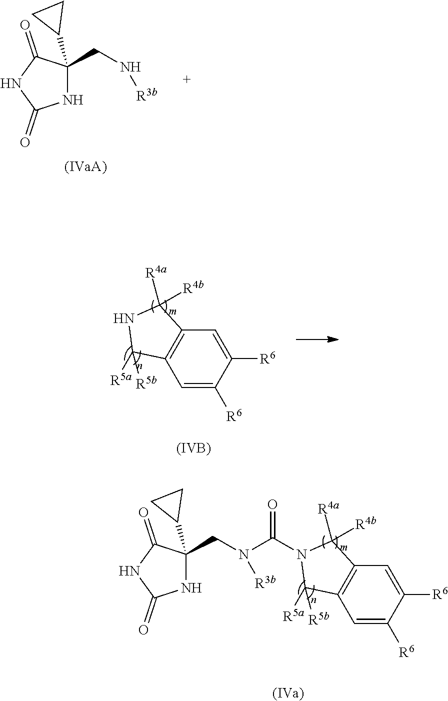

reacting a compound of formula (IVaA) with an acylation reagent to generate a reactive carbamate intermediate which can then be reacted with a compound of formula (IVB) to obtain the compound of formula (IVa), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride, but not limited to;

the acylation reagent is preferably 4-nitrophenyl chloroformate; and

R^{4a} to R^{5a}, R^{3b} to R^{5b}, R⁶, m and n are each as defined in formula (IVa).

The present disclosure provides a pharmaceutical composition comprising a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, and a pharmaceutically acceptable carrier.

The present disclosure relates to a method of inhibiting ADAMTS-5 and/or ADAMTS-4, comprising a step of administering to a subject in need thereof a therapeutically effective amount of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt thereof, solvate or prodrug thereof, or a pharmaceutical composition containing the compound.

The present disclosure relates to a method of preventing and/or treating ADAMTS-5 and/or ADAMTS-4 mediated diseases, comprising a step of administering to a subject in need thereof a therapeutically effective amount of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt thereof, solvate or prodrug thereof, or a pharmaceutical composition containing the compound. The present disclosure relates to a method of preventing and/or treating inflammatory conditions, and/or diseases involving degradation of cartilage and/or disruption of cartilage homeostasis, comprising a step of administering to a subject in need thereof a therapeutically effective amount of the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt thereof, solvate or prodrug thereof, or a pharmaceutical composition containing the compound.

The present disclosure relates to a method of preventing and/or treating arthritis, comprising a step of administering to a subject in need thereof a therapeutically effective amount of the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt thereof, solvate or prodrug thereof, or a pharmaceutical composition containing the compound; preferably, wherein arthritis is selected from the group consisting of rheumatoid arthritis, psoriatic arthritis, osteoarthritis and hypertrophic arthritis.

In another aspect, the present disclosure also relates to use of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for the inhibition of ADAMTS-5 and/or ADAMTS-4.

In another aspect, the present disclosure also relates to use of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for the preventing and/or treating of ADAMTS-5 and/or ADAMTS-4 mediated diseases.

In another aspect, the present disclosure also relates to use of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for preventing and/or treating inflammatory conditions, and/or diseases involving degradation of cartilage and/or disruption of cartilage homeostasis.

In another aspect, the present disclosure also relates to use of a compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for preventing and/or treating arthritis; preferably, rheumatoid arthritis, psoriatic arthritis, osteoarthritis and hypertrophic arthritis.

The present disclosure further relates to the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, for use as a medicament.

The present disclosure also relates to the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, for use in inhibiting ADAMTS-5 and/or ADAMTS-4.

The present disclosure also relates to the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, for use in preventing and/or treating ADAMTS-5 and/or ADAMTS-4 mediated diseases.

The present disclosure also relates to the combination of the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, for use in preventing and/or treating inflammatory conditions, and/or diseases involving degradation of cartilage and/or disruption of cartilage homeostasis.

The present disclosure also relates to the combination of the compound of formula (I), (II), (IIa), (III), (IIIa), (IV), (IVa), Table A or Table B, or a tautomer, mesomer, racemate, enantiomer, diastereomer, or mixture thereof, or a pharmaceutically acceptable salt, solvate or prodrug thereof, or a pharmaceutical composition containing the compound, for use in preventing and/or treating arthritis; preferably, rheumatoid arthritis, psoriatic arthritis, osteoarthritis and hypertrophic arthritis.

The ADAMTS-5 and/or ADAMTS-4 mediated diseases are selected from inflammatory conditions, and/or diseases involving degradation of cartilage and/or disruption of cartilage homeostasis, and/or arthritis; wherein arthritis is preferably selected from the group consisting of rheumatoid arthritis, psoriatic arthritis, osteoarthritis and hypertrophic arthritis.

The term “inflammatory conditions” refers to the group of conditions including rheumatoid arthritis, osteoarthritis, juvenile idiopathic arthritis, psoriasis, psoriatic arthritis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g. complications after bypass surgery or chronic endotoxin states contributing to e.g. chronic cardiac failure), and related diseases involving cartilage, such as that of the joints. Particularly, the term refers to rheumatoid arthritis, osteoarthritis, allergic airway disease (e.g. asthma), chronic obstructive pulmonary disease (COPD) and inflammatory bowel diseases. More particularly, it refers to rheumatoid arthritis, and osteoarthritis (OA). Most particularly, it refers to osteoarthritis (OA).

The term “diseases involving degradation of cartilage and/or disruption of cartilage homeostasis” includes conditions such as osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amyloidosis, hydrarthrosis, periodical disease, rheumatoid spondylitis, endemic forms of arthritis like osteoarthritis deformans endemic, Mseleni disease and Handigodu disease; degeneration resulting from fibromyalgia, systemic lupus erythematosus, scleroderma and ankylosing spondylitis. Particularly, it refers to osteoarthritis (OA).

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

Suitable dosage forms include, but are not limited to, a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from the group consisting of sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water-soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The active ingredient and the dispersants or wetting agents, suspending agent or one or more preservatives can be prepared as a dispersible powder or granule suitable for the preparation of an aqueous suspension by adding water. Suitable dispersants or wetting agents and suspending agents are exemplified by those already mentioned above. Additional excipients, such as sweeteners, flavoring agents and colorants, can also be added. These compositions can be preserved by adding an antioxidant such as ascorbic acid.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS™ 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound or the type of pharmaceutically acceptable salt thereof can be verified by traditional therapeutic regimens.

### DETAILED DESCRIPTION OF THE DISCLOSURE

Unless otherwise stated, the terms used in the specification and claims take ordinary meanings as understood by those in the ordinary skill in the relevant art. Certain terms have the meanings described below.

“Alkyl” refers to a saturated aliphatic hydrocarbon group including C₁-C₁₂ (e.g. C₁, C₂, C₃, C₄, C₅, C₆, C₇, C₈, C₉, C₁₀, C₁₁ and C₁₂) straight chain and branched chain groups. Preferably an alkyl group is an alkyl having 1 to 8 carbon atoms, sometimes more preferably 1 to 6 (e.g. 1, 2, 3, 4, 5 and 6) carbon atoms, and sometime more preferably 1 to 4 carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. The alkyl group can be substituted or unsubstituted. When substituted, the substituent group(s) can be substituted at any available connection point, preferably the substituent group(s) is one or more, preferably one to five, and more preferably one to three, groups independently selected from the group consisting of halogen, alkoxy, alkenyl, alkynyl, alkylsulfo, alkylamino, thiol, hydroxy, nitro, cyano, amino, cycloalkyl, heterocyclyl, aryl, heteroaryl, cycloalkoxy, heterocyclyloxy, cycloalkylthio, heterocyclylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₁₂ alkenyl, more preferably C₂₋₈ alkenyl, sometimes more preferably C₂₋₆ alkenyl, and sometimes more preferable C₂₋₄ alkenyl. The alkenyl group can be substituted or unsubstituted. When substituted, the substituent group(s) is preferably one or more, preferably one to five, and more preferably one to three, group(s) independently selected from the group consisting of halogen, alkoxy, alkenyl, alkynyl, alkylsulfo, alkylamino, thiol, hydroxy, nitro, cyano, amino, cycloalkyl, heterocyclyl, aryl, heteroaryl, cycloalkoxyl, heterocyclyloxy, cycloalkylthio, heterocyclylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butyryl etc., preferably C₂₋₁₂ alkynyl, more preferably C₂₋₆ alkynyl, sometimes more preferably C₂₋₆ alkynyl, and sometimes more preferable C₂₋₄ alkynyl. The alkynyl group can be substituted or unsubstituted. When substituted, the substituent group(s) is preferably one or more, preferably one to five, and more preferably one to three, group(s) independently selected from the group consisting of alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkylene” refers to a saturated linear or branched divalent aliphatic hydrocarbon group, derived by removing two hydrogen atoms from the same carbon atom or two different carbon atoms of the parent alkane. The straight or branched chain group containing 1 to 12 (e.g. 1, 2, 3, 4, 5, 6, 7, 8, 9, 10, 11 and 12) carbon atoms, preferably has 1 to 8 carbon atoms, more preferably 1 to 6 (e.g. 1, 2, 3, 4, 5 and 6) carbon atoms, and sometimes more preferably 1 to 4 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably 1 to 5, and sometimes more preferably 1 to 3, group(s) independently selected from the group consisting of selected from alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkenylene” refers to an alkylene defined as above that has at least two carbon atoms and at least one carbon-carbon double bond, preferably C₂₋₁₂ alkenylene, more preferably C₂₋₈ alkenylene, sometimes more preferably alkenylene, and sometimes even more preferably C₂-4 alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably 1 to 5, and sometimes more preferably 1 to 3, group(s) independently selected from the group consisting of selected from alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, sometimes more preferably 3 to 8 (e.g. 3, 4, 5, 6, 7 and 8) carbon atoms, and sometimes even more preferably 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds. Preferably a spiro cycloalkyl is 6 to 14 membered, and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 3-membered/5-membered, 3-membered/6-membered, 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following groups:

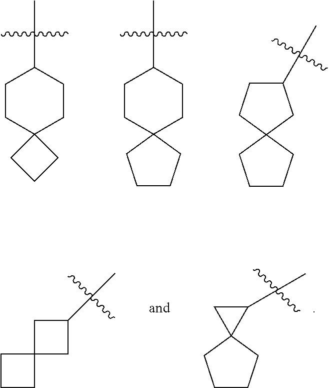 [SMILES: *C1CCC2(CCC2)CC1 ; *C1CCC2(CCCC2)CC1 ; *C1CCC2(CCCC2)C1 ; *C1CC2(CCC2)C1 ; *C1CC12CCCC2]

“Fused Cycloalkyl” refers to a 5 to 20 membered polycyclic hydrocarbon group, wherein each ring in the system shares an adjacent pair of carbon atoms with another ring, wherein one or more rings can contain one or more double bonds. Preferably, a fused cycloalkyl group is 6 to 14 membered, more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused cycloalkyl. Representative examples of fused cycloalkyls include, but are not limited to, the following groups:

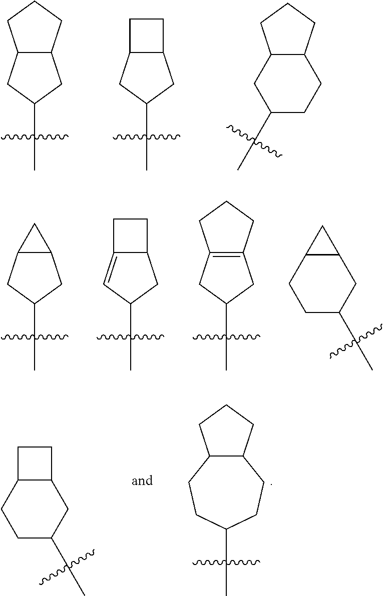

“Bridged Cycloalkyl” refers to a 5 to 20 membered polycyclic hydrocarbon group, wherein every two rings in the system share two disconnected carbon atoms. The rings can have one or more double bonds. Preferably, a bridged cycloalkyl is 6 to 14 membered, and more preferably 7 to 10 (e.g. 7, 8, 9 and 10) membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

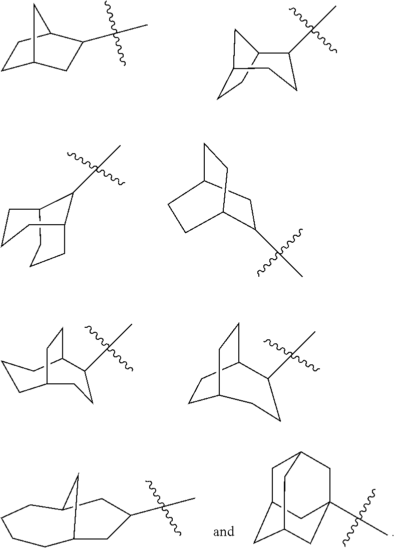

The cycloalkyl includes the cycloalkyl said above fused to the ring of an aryl, heteroaryl or heterocyclyl, wherein the ring bound to the parent structure is cycloalkyl. Representative examples include, but are not limited to indanyl, tetrahydronaphthalene, benzocycloheptyl and so on.

The cycloalkyl is optionally substituted or unsubstituted. When substituted, the substituent group(s) is preferably one or more, sometimes preferably 1 to 5, and sometimes more preferably 1 to 3, groups independently selected from the group consisting of alkyl, halogen, alkoxy, alkenyl, alkynyl, alkylsulfo, alkylamino, thiol, hydroxy, nitro, cyano, amino, cycloalkyl, heterocyclyl, aryl, heteroaryl, cycloalkoxyl, heterocyclyloxy, cycloalkylthio, heterocyclylthio and oxo group.

“Heterocyclyl” refers to a 3 to 20 membered saturated and/or partially unsaturated monocyclic or polycyclic hydrocarbon group having one or more heteroatoms selected from the group consisting of N, O, S, S(═O) and S(O)₂ as ring atoms, but excluding —O—O—, —O—S— or —S—S— in the ring, the remaining ring atoms being C. Preferably, heterocyclyl is a 3 to 12 (e.g. 3, 4, 5, 6, 7, 8, 9, 10, 11 or 12) membered having 1 to 4 (e.g. 1, 2, 3 or 4) heteroatoms; more preferably a 3 to 10 (e.g. 3, 4, 5, 6, 7, 8, 9 or 10) membered having 1 to 3 (e.g. 1, 2 or 3) heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more heteroatoms selected from the group consisting of N, O, S, S(═O) and S(O)₂ as ring atoms, the remaining ring atoms being C, wherein one or more rings can contain one or more double bonds. Preferably a spiro heterocyclyl is 6 to 14 membered, and more preferably 7 to 10 (e.g. 7, 8, 9 and 10) membered. According to the number of common spiro atoms, Spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 3-membered/5-membered, 3-membered/6-membered, 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following groups:

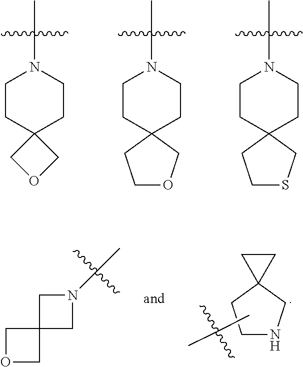

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, and wherein said rings have one or more heteroatoms selected from the group consisting of N, O, S, S(═O) and S(O)₂ as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 3-membered/4-membered, 3-membered/5-membered, 3-membered/6-membered, 4-membered/4-membered, 4-membered/5-membered, 4-membered/6-membered, 5-membered/4-membered, 5-membered/5-membered, 5-membered/6-membered, 6-membered/3-membered, 6-membered/4-membered, 6-membered/5-membered or 6-membered/6-membered bicyclic fused heterocyclyl. Representative examples of fused heterocyclyl include, but are not limited to, the following groups:

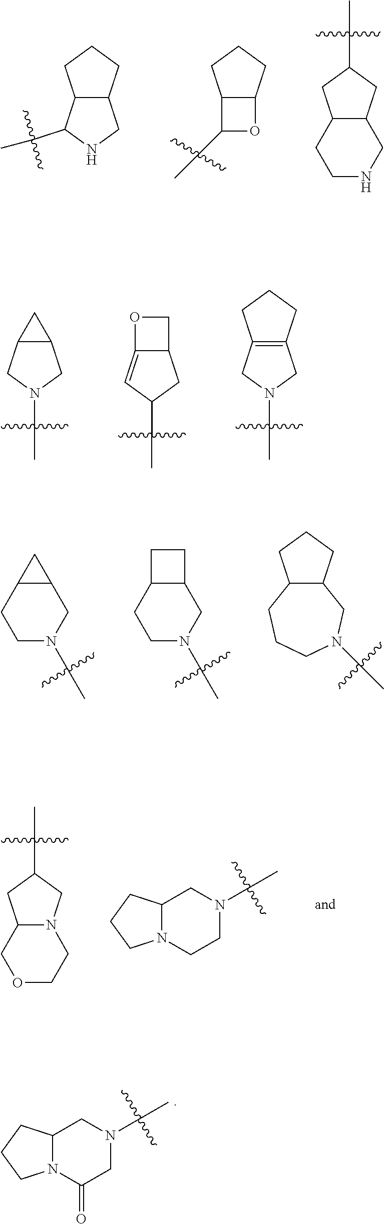

“Bridged Heterocyclyl” refers to a 5 to 14 membered polycyclic heterocyclyl group, wherein every two rings in the system share two disconnected atoms, the rings can have one or more double bonds, and the rings have one or more heteroatoms selected from the group consisting of N, O, S, S(═O) and S(O)₂ as ring atoms, the remaining ring atoms being C. Preferably a bridged heterocyclyl is 6 to 14 membered, and more preferably 7 to 10 (e.g. 7, 8, 9 and 10) membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

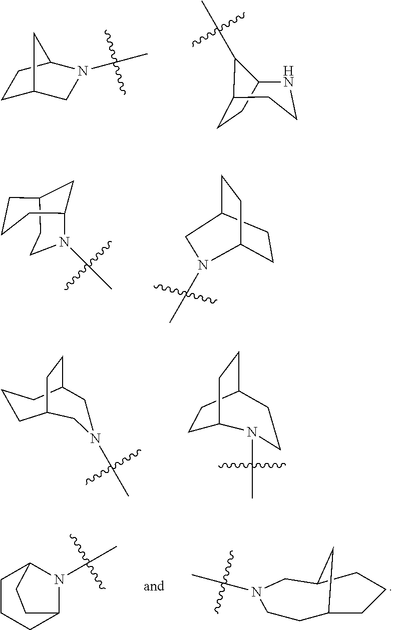

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

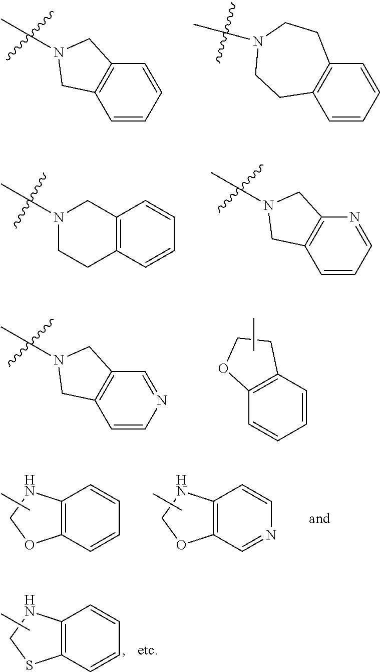

The heterocyclyl is optionally substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl includes the aryl said above which fused to the ring of heteroaryl, heterocyclyl or cycloalkyl, wherein the ring bound to parent structure is aryl. Representative examples include, but are not limited to, the following groups:

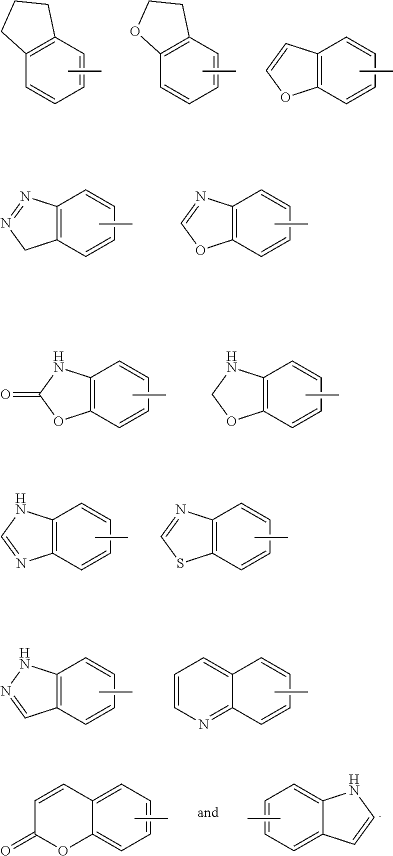

The aryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Heteroaryl” refers to an aryl system having 1 to 4 (e.g. 1, 2, 3 or 4) heteroatoms selected from the group consisting of O, S and N as ring atoms and having 5 to 14 annular atoms. Preferably a heteroaryl is 5- to 10-(e.g. 5, 6, 7, 8, 9 or 10) membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

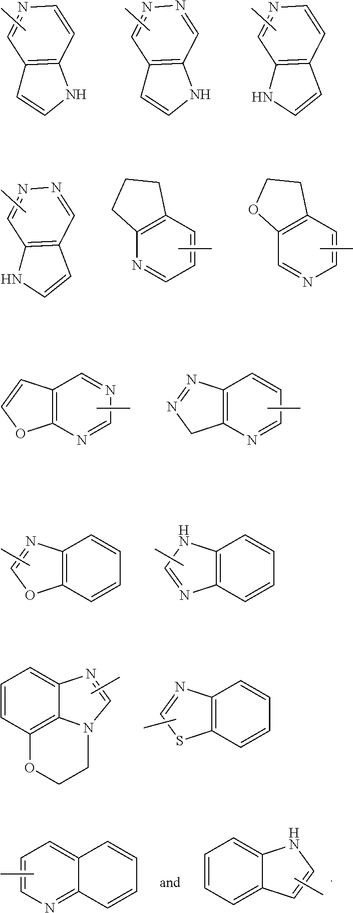

The heteroaryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

“Alkoxy” refers to both an —O-(alkyl) group, wherein the alkyl is defined as above. Representative examples include, but are not limited to, methoxy, ethoxy, propoxy, butoxy, and the like. The alkoxyl can be substituted or unsubstituted. When substituted, the substituent is preferably one or more, sometimes preferably 1 to 5, and sometimes more preferably 1 to 3, groups independently selected from the group consisting of alkenyl, alkynyl, alkoxy, alkylsulfo, alkylamino, halogen, thiol, hydroxy, nitro, cyano, cycloalkyl, heterocyclyl, aryl, heteroaryl, cycloalkoxyl, heterocyclyloxy, cycloalkylthio and heterocyclylthio.

The above-mentioned cycloalkyl, heterocyclyl, aryl and heteroaryl groups contain one monovalent residue derived from the removal of one hydrogen atom from the parent ring atom, or one divalent residue derived from the removal of two hydrogen atoms from the same or different ring atoms of the parent, namely “divalent cycloalkyl”, “divalent heterocyclyl”, “arylene”, and “heteroarylene”. “Bond” refers to a covalent bond using a sign of “—”. “Hydroxyalkyl” refers to an alkyl group substituted by one or more hydroxy group(s), wherein alkyl is as defined above.

“Hydroxy” refers to an —OH group.

“haloalkyl” refers to an alkyl group substituted by one or more halogen(s), wherein alkyl is as defined above.

“deuterated alkyl” refers to an alkyl group substituted by one or more deuterium atom(s), wherein alkyl is as defined above.

“thiol” refers to a —SH group. “alkylthio” refers to an alkyl-S— group, wherein alkyl is as defined above.

“haloalkylthio” refers to a haloalkyl-S— group, wherein haloalkyl is as defined above.

“cycloalkoxyl” refers to a cycloalkyl-O—, wherein cycloalkyl is as defined above.

“heterocylyloxy” refers to a heterocyclyl-O—, wherein heterocyclyl is as defined above.

“cycloalkylthio” refers to a cycloalkyl-S—, wherein cycloalkyl is as defined above.

“heterocyclylthio” refers to a heterocyclyl-S—, wherein heterocyclyl is as defined above.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(═O)OH group.

“Carboxylate” refers to a —C(═O)O(alkyl), —C(═O)O(cycloalkyl), —OC(═O)(alkyl) or —OC(═O)(cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity. The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N, N-dimethylaniline, N-methylpiperidine, and N-methylmorpholine.

As a person skilled in the art would understand, the compounds of formula (I), (II), (IIa), (III), (IIIa), (IV), (IVa), Table A or Table B, or pharmaceutically acceptable salts thereof disclosed herein may exist in prodrug or solvate forms, which are all encompassed by the present disclosure.

The term “solvate,” as used herein, means a physical association of a compound of this disclosure with one or more, preferably one to three, solvent molecules, whether organic or inorganic. This physical association includes hydrogen bonding. In certain instances, the solvate will be capable of isolation, for example, when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

“Prodrug” refers to compounds that can be transformed in vivo to yield the active parent compound under physiological conditions, such as through hydrolysis in blood. Common examples include, but are not limited to, ester and amide forms of a compound having an active form bearing a carboxylic acid moiety. Amides and esters of the compounds of the present disclosure may be prepared according to conventional methods. In particular, in the present disclosure, a prodrug may also be formed by acylation of an amino group or a nitrogen atom in a heterocyclyl ring structure, which acyl group can be hydrolyzed in vivo. Such acyl group includes, but is not limited to, a C₁-C₆ acyl, preferably C₁-C₄ acyl, and more preferably C₁-C₂ (formyl or acetyl) group, or benzoyl.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

As used herein, the singular forms “a”, “an”, and “the” include plural reference, and vice versa, unless the context clearly dictates otherwise.

When the term “about” is applied to a parameter, such as pH, concentration, temperature, or the like, it indicates that the parameter can vary by +10%, and sometimes more preferably within ±5%. As would be understood by a person skilled in the art, when a parameter is not critical, a number is often given only for illustration purpose, instead of being limiting.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (D) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents.

The compound of the present disclosure, any atom not specifically designated as a specific isotope means any stable isotope of that atom. Unless otherwise stated, when a position is specifically designated as “H” or “hydrogen”, the position should be understood as having hydrogen according to its natural abundance isotopic composition. Likewise, unless otherwise specified, when a position is specifically designated as “D” or “deuterium”, the position should be understood as deuterium having an abundance of at least 3000 times greater than the natural abundance of deuterium (which is 0.015%) (that is, at least 45% incorporation of deuterium). Exampled compounds have deuterium with an abundance of at least 1000 times greater than the natural abundance of deuterium (that is, at least 15% incorporation of deuterium), at least 2000 times greater than the natural abundance of deuterium (that is, at least 30% incorporation of deuterium), at least 3000 times greater than the natural abundance of deuterium (that is, at least 45% incorporation of deuterium), at least 3340 times greater than the natural abundance of deuterium (that is, at least 50.1% incorporation of deuterium), at least 3500 times greater than the natural abundance of deuterium (that is, at least 52.5% incorporation of deuterium), at least 4000 times greater than the natural abundance of deuterium (that is, at least 60% incorporation of deuterium), at least 4500 times greater than the natural abundance of deuterium (that is, at least 67.5% incorporation of deuterium), at least 5000 times greater than the natural abundance of deuterium (that is, at least 75% incorporation of deuterium), at least 5,500 times greater than the natural abundance of deuterium (that is, at least 82.5% incorporation of deuterium), at least 6000 times greater than the natural abundance of deuterium (that is, at least 90% incorporation of deuterium) at least 6333.3 times greater than the natural abundance of deuterium (that is, at least 95% incorporation of deuterium), at least 6466.7 times greater than the natural abundance of deuterium (that is, at least 97% incorporation of deuterium), at least 6600 times greater than the natural abundance of deuterium (that is, at least 99% incorporation of deuterium), at least 6633.3 times greater than the natural abundance of deuterium (that is, at least 99.5% incorporation of deuterium) or a higher abundance of deuterium.

### Synthesis Method of the Compounds

In order to complete the purpose of the disclosure, the present disclosure applies, but is not limited to, the following technical solution:

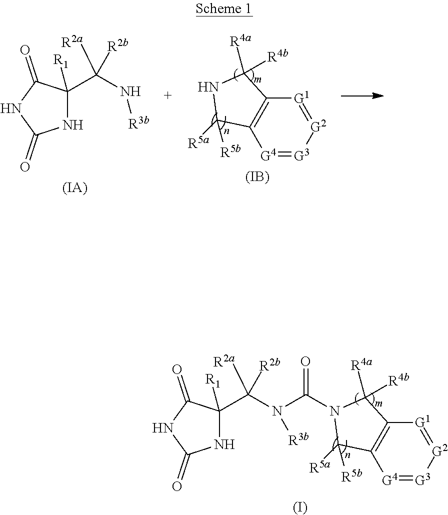

A preparation process of a compound of formula (I), or a tautomer, mesomer, racemate, enantiomer, diastereomer, or mixture thereof, or a pharmaceutically acceptable salt thereof, solvate or prodrug thereof comprising a step of:

reacting a compound of formula (IA) with an acylation reagent to create a reactive carbamate intermediate which can then be reacted with a compound of formula (IB) to obtain the compound of formula (I) or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

G¹, G², G³, G⁴, R¹, R^{2a}, R^{4a} to R^{5a}, R^{2b} to R^{5b}, n and m are each as defined in formula (I).

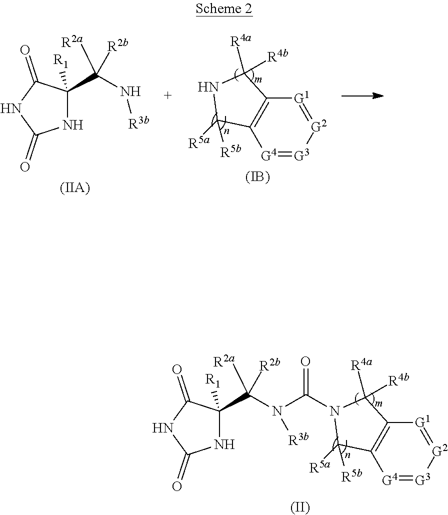

A preparation process of a compound of formula (I), or a tautomer, mesomer, racemate, enantiomer, diastereomer, or mixture thereof, or a pharmaceutically acceptable salt thereof, solvate or prodrug thereof comprising a step of:

reacting a compound of formula (IIA) with an acylation reagent to create a reactive carbamate intermediate which can then be reacted with a compound of formula (IB) to obtain the compound of formula (II) or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

G¹, G², G³, G⁴, R¹, R^{2a}, R^{4a} to R^{5a}, R^{2b} to R^{5b}, n and m are each as defined in formula (II).

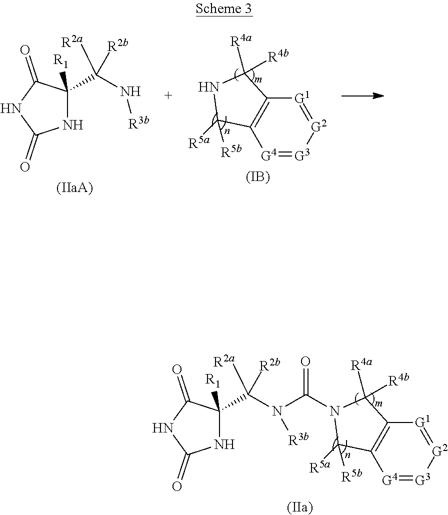

A preparation process of a compound of formula (IIa), or a tautomer, mesomer, racemate, enantiomer, diastereomer, or mixture thereof, or a pharmaceutically acceptable salt thereof, solvate or prodrug thereof comprising a step of:

reacting a compound of formula (IIaA) with an acylation reagent to create a reactive carbamate intermediate which can then be reacted with a compound of formula (IB) to obtain the compound of formula (IIa) or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

G¹, G², G³, G⁴, R¹, R^{2a}, R^{4a} to R^{5a}, R^{2b} to R^{5b}, n and m are each as defined in formula (IIa).

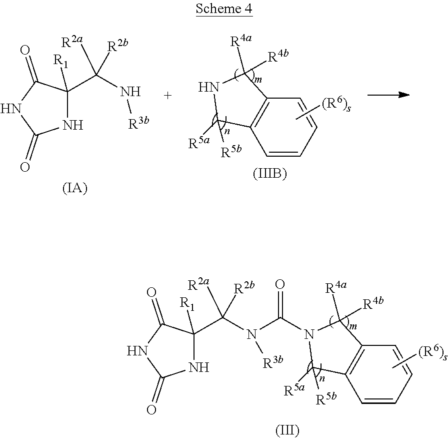

A preparation process of a compound of formula (III), or a tautomer, mesomer, racemate, enantiomer, diastereomer, or mixture thereof, or a pharmaceutically acceptable salt thereof, solvate or prodrug thereof comprising a step of:

reacting a compound of formula (IA) with an acylation reagent to create a reactive carbamate intermediate which can then be reacted with a compound of formula (IIIB) to obtain the compound of formula (III) or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

R¹, R^{2a}, R^{4a} to R^{5a}, R^{2b} to R^{5b}, R⁶, n, m and s are each as defined in formula (III).

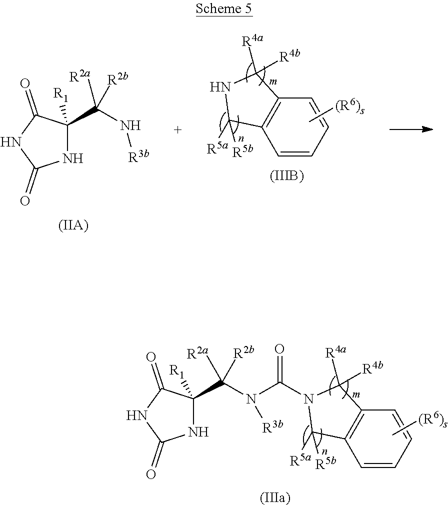

A preparation process of a compound of formula (IIIa), or a tautomer, mesomer, racemate, enantiomer, diastereomer, or mixture thereof, or a pharmaceutically acceptable salt thereof, solvate or prodrug thereof comprising a step of:

reacting a compound of formula (IIA) with an acylation reagent to create a reactive carbamate intermediate which can then be reacted with a compound of formula (IIIB) to obtain the compound of formula (IIIa) or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

R¹, R^{2a}, R^{4a} to R^{5a}, R^{2b} to R^{5b}, R⁶, n, m and s are each as defined in formula (IIIa).

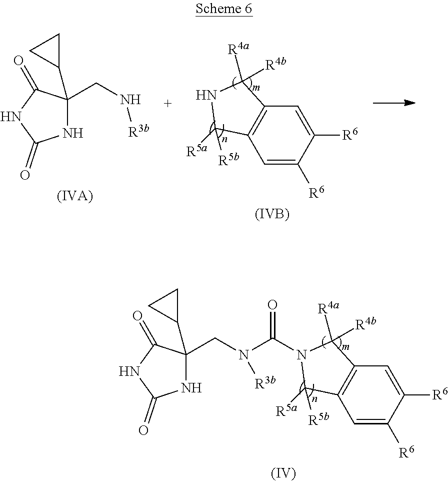

A preparation process of a compound of formula (IV), or a tautomer, mesomer, racemate, enantiomer, diastereomer, or mixture thereof, or a pharmaceutically acceptable salt thereof, solvate or prodrug thereof comprising the steps of:

reacting a compound of formula (IVA) with an acylation reagent to generate a reactive carbamate intermediate which can then be reacted with a compound of formula (IVB) to obtain the compound of formula (IV), or a tautomer, mesomer, racemate, enantiomer, diastereomer, or mixture thereof, or a pharmaceutically acceptable salt thereof, solvate or prodrug thereof;

wherein:

pharmaceutically acceptable salt thereof preferably is hydrochloride; and

R^{4a} to R^{5a}, R^{3b} to R^{5b}, R⁶, m and n are each as defined in formula (IV).

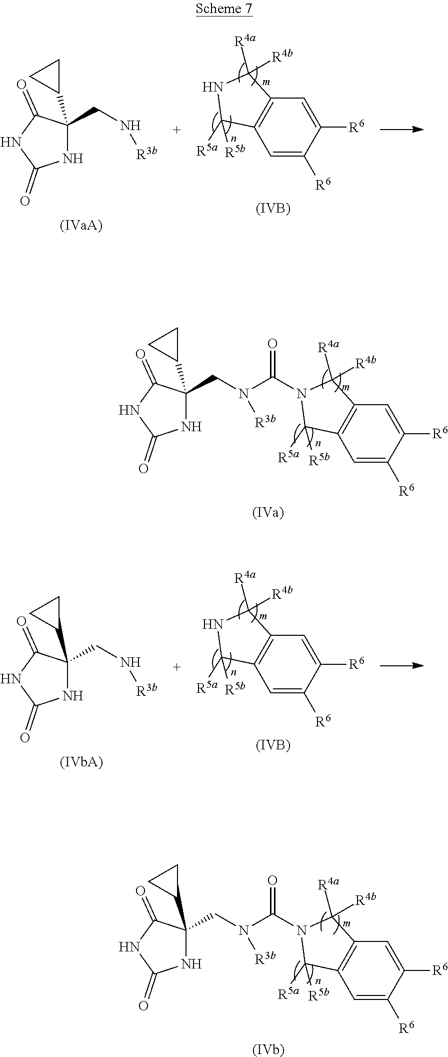

A preparation process of a compound of formula (IVa) or (IVb), or a tautomer, mesomer, racemate, enantiomer, diastereomer, or mixture thereof or a pharmaceutically acceptable salt thereof, solvate or prodrug thereof comprising the steps of:

reacting a compound of formula (IVaA) with an acylation reagent to generate a reactive carbamate intermediate which can then be reacted with a compound of formula (IVB) to obtain the compound of formula. (IVa), or a tautomer, mesomer, racemate, enantiomer, diastereomer, or mixture thereof, or a pharmaceutically acceptable salt thereof, solvate or prodrug thereof;

reacting a compound of formula (IVbA) with an acylation reagent to generate a reactive carbamate intermediate which can then be reacted with a compound of formula (IVB) to obtain the compound of formula (IVb), or a tautomer, mesomer, racemate, enantiomer, diastereomer, or mixture thereof, or a pharmaceutically acceptable salt thereof, solvate or prodrug thereof;

wherein;

pharmaceutically acceptable salt thereof preferably is hydrochloride; and

R^{4a} to R^{5a}, R^{3b} to R^{5b}, R⁶, m and n are each as defined in formula (IV).

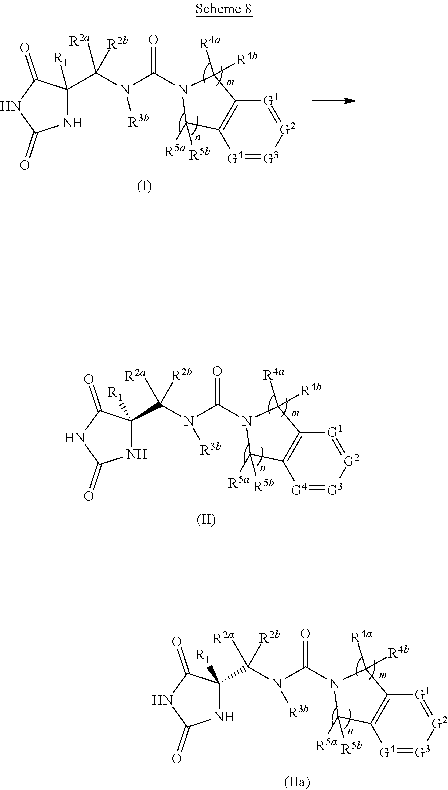

A preparation process of a compound of formula (II) or (IIa), or a tautomer, mesomer, racemate, enantiomer, diastereomer, or mixture thereof, or a pharmaceutically acceptable salt thereof, solvate or prodrug thereof comprising a step of:

Formula (I) or a tautomer, mesomer, racemate, enantiomer, diastereomer, or mixture thereof, or a pharmaceutically acceptable salt, solvate or prodrug thereof, was chiral separated to give Formula (II) and (IIa) or a tautomer, mesomer, racemate, enantiomer, diastereomer, or mixture thereof, or a pharmaceutically acceptable salt, solvate or prodrug thereof;

wherein:

G¹, G², G³, G⁴, R^{2a}, R^{4a} to R^{5a}, R^{2b} to R^{5b}, n and m are each as defined in formula (I).

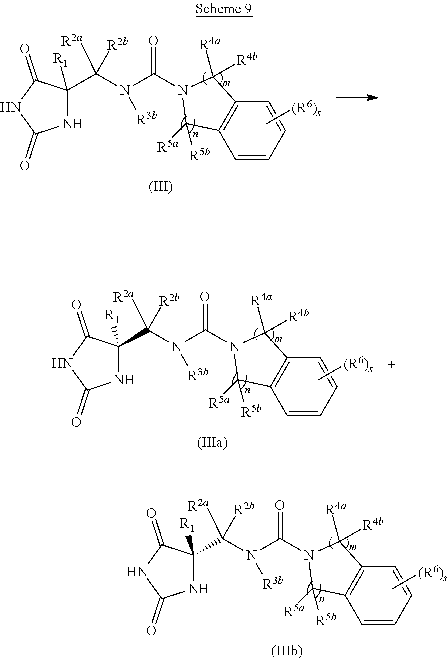

A preparation process of a compound of formula (IIIa) or (IIIb), or a tautomer, mesomer, racemate, enantiomer, diastereomer, or mixture thereof, or a pharmaceutically acceptable salt thereof, solvate or prodrug thereof comprising a step of:

Formula (III) or a tautomer, mesomer, racemate, enantiomer, diastereomer, or mixture thereof, or a pharmaceutically acceptable salt, solvate or prodrug thereof, was chiral separated to give Formula (IIIa) and (IIIb) or a tautomer, mesomer, racemate, enantiomer, diastereomer, or mixture thereof, or a pharmaceutically acceptable salt, solvate or prodrug thereof;

wherein:

R¹, R^{2a}, R^{4a} to R^{5a}, R^{2b} to R^{5b}, R⁶, n, s and m are each as defined in formula (III).

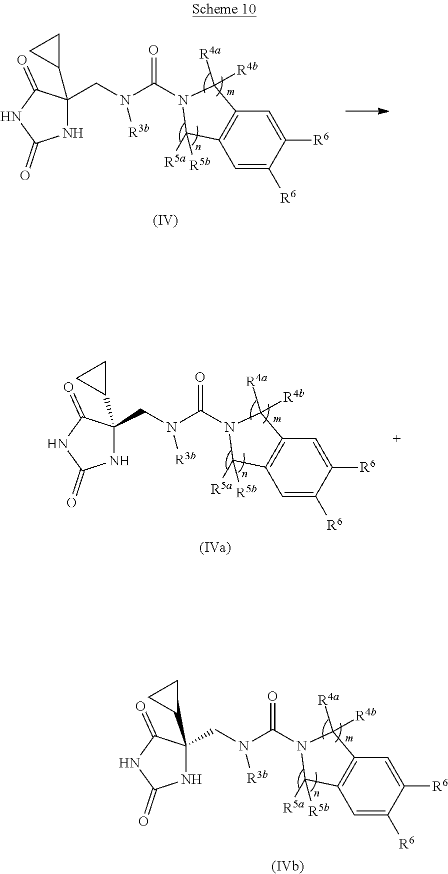

A preparation process of a compound of formula (IVa) or (IVb), or a tautomer, mesomer, racemate, enantiomer, diastereomer, or mixture thereof, or a pharmaceutically acceptable salt thereof, solvate or prodrug thereof comprising a step of:

Formula (IV) or a tautomer, mesomer, racemate, enantiomer, diastereomer, or mixture thereof, or a pharmaceutically acceptable salt, solvate or prodrug thereof, was chiral separated to give Formula (IVa) and (IVb) or a tautomer, mesomer, racemate, enantiomer, diastereomer, or mixture thereof, or a pharmaceutically acceptable salt, solvate or prodrug thereof;

wherein:

R^{4a} to R^{5a}, R^{3b} to R^{5b}, R⁶, n and m are each as defined in formula (IV).

The base includes organic bases and inorganic bases, wherein the organic base includes, but is not limited to, triethylamine, N,N-disopropylethylamine, n-butyllithium, lithium diisopropylamide, potassium acetate, sodium tert-butoxide and potassium tert-butoxide, and wherein the inorganic base includes, but is not limited to, magnesium chloride, sodium hydride, potassium phosphate, sodium carbonate, potassium carbonate, cesium carbonate and N-(3-Dimethylaminopropyl)-N′-ethylcarbodiimide hydrochloride (EDCI).

The acylation agent includes, but is not limited to, 4-nitrophenyl carbonochloridate (also named 4-nitrophenyl chloroformate), CO₂, (Cl₃CO)₂CO, ClCOOCH₂C₆H₅, diimidazolyl ketone, C₆H₅OCOCl and COCl₂, preferably 4-nitrophenyl carbonochloridate.

The reaction is preferably in solvent, wherein solvent used herein includes, but is not limited to, acetic acid, methanol, ethanol, toluene, tetrahydrofuran, dichloromethane, dimethylsulfoxide, 1,4-dioxane, water, N,N-dimethylformamide, and the mixture thereof.

The following examples serve to illustrate the disclosure, but the examples should not be considered as limiting the scope of the disclosure. If specific conditions for the experimental method are not specified in the examples of the present disclosure, they are generally in accordance with conventional conditions or recommended conditions of the raw materials and the product manufacturer. The reagents without a specific source indicated are commercially available, conventional reagents.

The structures of the compounds were identified by nuclear magnetic resonance (NMR) and/or mass spectrometry (MS). NMR was determined by a Bruker AVANCE II (or III)-400 MHz. The solvents are deuterated-dimethyl sulfoxide (DMSO-d₆), deuterated-chloroform (CDCl₃) and deuterated-methanol (CD₃OD) with tetramethylsilane (TMS) as an internal standard. NMR chemical shifts (δ) are given in 10⁻⁶ (ppm).

LC/MS (ESI) analyses were performed on a Shimadzu LCMS2020 equipped with a Sunfire C18 (5 μm50×4.6 mm) column, Waters UPLC-QDa equipped with an ACQUITY UPLC® BEH (2.1*50 mm 1.7 um) column, Agilent Agilent6120 equipped with a Xbridge C18 (5 μm50×4.6 mm) column.

HPLC analyses were performed on an Agilent 1200DAD equipped with a Sunfire C18 (5 μm150×4.6 mm) column and Shimadzu UFLC equipped with an Xbridge C18 (5 μm150×4.6 mm) column.

Chiral HPLC analyses were performed on a Waters-UPC² instrument.

The known raw materials of the present disclosure were prepared by the conventional synthesis methods in the art, or purchased from. Aldrich Chemical Company, Fisher Scientific or Combi-Blocks, etc.

Unless otherwise stated, the reactions were carried out under nitrogen atmosphere.

Unless otherwise stated, the reaction temperature in the reactions refers to room temperature, and the range of the temperature was 20° C. to 30° C.

The reaction process was monitored by LC-MS or thin layer chromatography (TLC), and the developing solvent system includes: A: dichloromethane and methanol, B: hexane and ethyl acetate, etc. The ratio of the volume of the solvent was adjusted according to the polarity of the compounds. The elution system for purification of the compounds by column chromatography, thin layer chromatography and CombiFlash flash rapid preparation instrument includes: A: dichloromethane and methanol, B: hexane and ethyl acetate, etc. The ratio of the volume of the solvent was adjusted according to the polarity of the compounds, and sometimes a small amount of basic reagent such as ammonia or acidic reagent such as acetic acid was added.

Prep-HPLC was performed on Shimadzu (LC-20AD, SPD20A) Preparative HPLC (Phenomenex Gemini-NX 5 μM C18 21.2×100 mm column), Waters 2767 equipped with a Sunfire Pre C18 (10 μm19×250 mm) column and Waters 2767-QDa equipped with an Xbridge Pre C18 (10 μm19×250 mm) column instrument.

Pre-SFC was performed on a Waters-SFC80 equipped with Daciel AD/OD/OJ/IC/IA/ID (10 μm20×250 mm) column instrument.

Column chromatography was performed using silica gel 100-200 mesh or 200-300 mesh (purchased from YuCheng Chemical Shanghai, Co., Ltd) as the solid support.

CombiFlash was performed on systems from Teledyne ISCO or Agela Technologies.

The following abbreviations are used:

DIPEA (or DIEA) is N, N-diisopropylethylamine,

EDCI is N-(3-Dimethylaminopropyl)-N′-ethylcarbodiimide hydrochloride,

HOBt is 1-Hydroxybenzotriazole hydrate,

HATU is O-(7-Azabenzotriazol-1-yl)-N, N, N′,N′-tetramethyluronium hexafluorophosphate,

HBTU is O-(Benzotriazol-1-yl)-N, N, N′,N′-tetramethyluronium hexafluorophosphate,

AIBN is 2,2′-Azobis(2-methylpropionitrile),

NBS is N-bromosuccinimide,

NaIO₄ is sodium periodate,

OsO₄ is osmium tetroxide,

DAST is diethylaminosulfur trifluoride,

HCl is hydrogen chloride,

LDA is lithium diisopropylamide,

TEA is triethylamine,

DCM is dichloromethane,

DMF is N, N-dimethylformamide,

EtOAc is ethyl acetate,

EtOH is ethanol,

MeCN or ACN is acetonitrile,

THF is Tetrahydrofuran,

NMR is nuclear magnetic resonance,

MS is mass spectroscopy with (+) referring to the positive mode which generally gives a M+1 (or M+H) absorption where M=the molecular mass.

Prep HPLC is Preparative high performance liquid chromatography.

SFC is Supercritical fluid chromatography.

SGC is silica gel chromatography.

### Intermediate 1 (Int-1)

### 5-(aminomethyl)-5-cyclopropylimidazolidine-2,4-dione hydrochloride

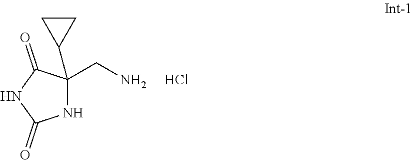 [SMILES: NCC1(C2CC2)NC(=O)NC1=O ; Cl]

### Intermediate 1a (Int-1A)

### (S)-5-(aminomethyl)-5-cyclopropylimidazolidine-2,4-dione hydrochloride Int-1A

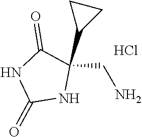 [SMILES: NC[C@]1(C2CC2)NC(=O)NC1=O ; Cl]

### Intermediate 1b (Int-1B)

### (R)-5-(aminomethyl)-5-cyclopropylimidazolidine-2,4-dione hydrochloride Int-1B

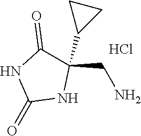 [SMILES: NC[C@@]1(C2CC2)NC(=O)NC1=O ; Cl]

### Synthetic Route

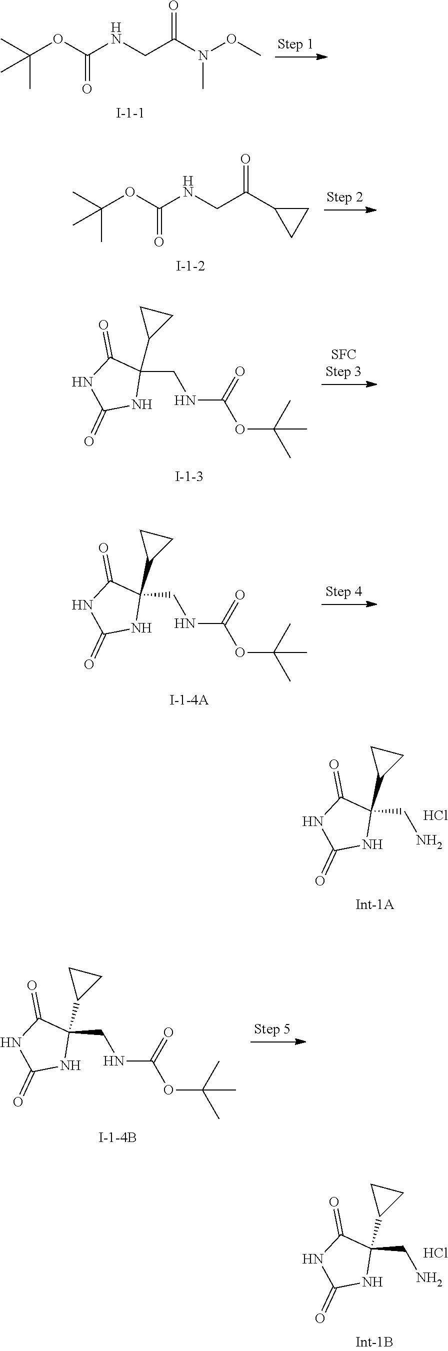

### Step 1

### Tert-butyl (2-cyclopropyl-2-oxoethyl)carbamate I-1-2

The solution of tert-butyl (2-(methoxy(methyl)amino)-2-oxoethyl)carbamate I-1-1 (14 g, 64.15 mmol) in THF (30 mL) was cooled to 10° C. before cyclopropylmagnesium bromide (26.99 g, 192.44 mmol) was added drop-wise. Then the reaction was stirred at 10° C. for 2 hrs. TLC showed the reaction completed, 1N HCl was added to quench the reaction and the mixture was extracted with EtOAc, the combined organic phase was dried over Na₂SO₄, filtered and concentrated. The crude product was purified by silica gel chromatography column to give I-1-2 (8 g, 40.15 mmol, 62.6% yield).

### Step 2

### Tert-butyl ((4-cyclopropyl-2,5-dioxoimidazolidin-4-yl)methyl)carbamate I-1-3

The mixture of I-1-2 (10.8 g, 54.20 mmol) and (NH₄)₂CO₃ (26.02 g, 271.02 mmol), KCN (7.50 g, 108.41 mmol) in methanol (100 mL), water (50 mL) was sealed and heated to 80° C. for 16 hrs. After the reaction completed, the reaction was diluted with water (350 mL) and EtOAc (200 mL), layers were separated and the water phase was extracted with EtOAc, the combined organic phases were dried over Na₂SO₄, filtered and concentrated. The residue was triturated with hexane (15 mL) and EtOAc (10 mL) for 1 hour and filtered to give I-1-3 (5.7 g, 21.17 mmol, 39.05% yield).

### Step 3

### Tert-butyl (S)-((4-cyclopropyl-2,5-dioxoimidazolidin-4-yl)methyl)carbamate I-1-4A & Tert-butyl (R)-((4-cyclopropyl-2,5-dioxoimidazolidin-4-yl)methyl)carbamate I-1-4B

The racemic mixture I-1-3 (5.7 g, 21.17 mmol) was chirally separated (using a chiral column. 11×33 cm chiralpak AD^{R}, 20 um; Flow Rate/detection: 80 mL/min/230 nm; Mobile phase: methanol) to give:

I-1-4A (2.1 g, 36.8% yield).

¹H NMR (400 MHz, DMSO-d₆): δ 10.54 (brs, 1H), 7.35 (s, 1H), 6.78 (brs, 1H), 3.32-3.28 (m, 2H), 1.37 (s, 9H), 1.06-1.03 (m, 1H), 0.43-0.37 (m, 2H), 0.31-0.29 (m, 1H), 0.11-0.08 (m, 1H).

Chiral HPLC: Rt: 1.808 min, ee: 100%.

LCMS: m/z (ESI): 292.1[M+Na]

I-1-4B (2.3 g, 40.3% yield).

¹H NMR (400 MHz, DMSO-d₆): δ 10.54 (brs, 1H), 7.35 (s, 1H), 6.78 (brs, 1H), 3.33-3.28 (m, 2H), 1.37 (s, 9H), 1.06-1.03 (m, 1H), 0.43-0.39 (m, 2H), 0.32-0.28 (m, 1H), 0.11-0.08 (m, 1H).

Chiral HPLC: Rt: 2.187 min, ee: 98.56%.

LCMS: m/z (ESI): 292.1 [M+Na]

### Step 4

### (S)-5-(aminomethyl)-5-cyclopropylimidazolidine-2,4-dione hydrochloride Int-1A

The solution of I-1-4A (2.1 g, 8.54 mmol) and HCl/dioxane (5 g, 8.54 mmol) in methanol (5 mL) was stirred at 20° C. for 1 hr. TLC showed the reaction completed and the precipitate was filtered to give Int-1A (1.4 g, 87.5% yield).

¹H NMR (400 MHz, DMSO-d₆): δ 10.78 (s, 1H), 8.26 (brs, 3H), 7.82 (s, 1H), 3.13-3.09 (m, 1H), 2.93-2.89 (m, 1H), 1.04-0.97 (m, 1H), 0.46-0.32 (m, 2H), 0.29-0.23 (m, 1H), 0.03-0.00 (m, 1H).

LCMS: m/z (ESI): 170.3 [M+H]

### Step 5

### (R)-5-(aminomethyl)-5-cyclopropylimidazolidine-2,4-dione hydrochloride Int-1B

The solution of I-1-4B (2.3 g, 8.54 mmol) and HCl/dioxane (5 g, 8.54 mmol) in methanol 5 mL) was stirred at 20° C. for 1 hr. TLC showed the reaction completed and the precipitate was filtered to give Int-1B (1.52 g, 86.54% yield).

¹H NMR (400 MHz, DMSO-d₆): δ 10.93 (s, 1H), 8.39 (brs, 3H), 7.96 (s, 1H), 3.26 (d, 1H), 3.05 (d, 1H), 1.18-1.13 (m, 1H), 0.59-0.55 (m, 2H), 0.43-0.39 (m, 1H), 0.16-0.12 (m, 1H).

LCMS: m/z (ESI): 170.2 [M+H]

Int-1 was prepared from I-1-3 using similar procedure as step 4.

### Example 1

### 5,6-dichloro-N-((4-cyclopropyl-2,5-dioxoimidazolidin-4-yl)methyl)isoindoline-2-carboxamide 1

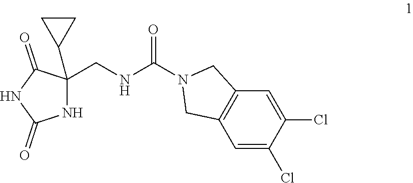 [SMILES: O=C1NC(=O)C(CNC(=O)N2Cc3cc(Cl)c(Cl)cc3C2)(C2CC2)N1]

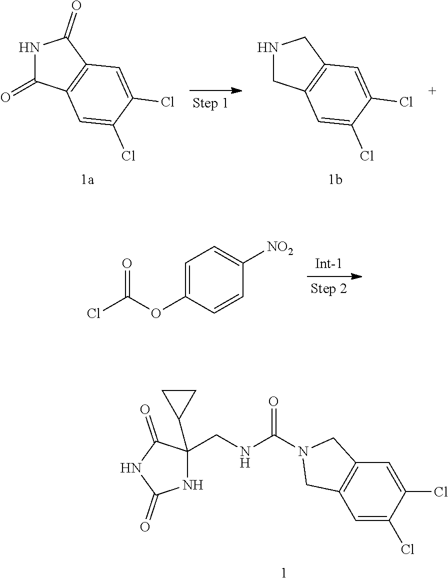 [SMILES: O=C1NC(=O)c2cc(Cl)c(Cl)cc21 ; Clc1cc2c(cc1Cl)CNC2 ; O=C(Cl)Oc1ccc([N+](=O)[O-])cc1 ; O=C1NC(=O)C(CNC(=O)N2Cc3cc(Cl)c(Cl)cc3C2)(C2CC2)N1]

### Step 1

### 5,6-dichloroisoindoline (1b)

To a solution of 4,5-dichlorophthalamide 1a (5 g, 23 mmol) in THF (15 mL) was added borane-tetrahydrofuran (1 M, 100 mL) dropwise under N₂. The resulting mixture was stirred at 60° C. for 24 h. The reaction mixture was cooled to ambient temperature and quenched with MeOH (6 ml) until the bubbling ceased. Then 4N HCl in water (20 ml) was added and the mixture was heated at 80° C. for 3 h. After cooled down to RT and 5N KOH was added to adjust pH to 7. The mixture was concentrated under reduced pressure and the residue was purified by silica-gel column (DCM:MeOH (2% NH₄OH) 10:1) to afford 5,6-dichloroisoindoline 1b (3 g, 70% yield).

### Step 2

### 5,6-dichloro-N-((4-cyclopropyl-2,5-dioxoimidazolidin-4-yl)methyl)isoindoline-2-carboxamide 1

A solution of compound Int-1 (15 mg) in THF (3 ml) was added over 15 mins to a stirring solution of 4-nitrophenyl chloroformate (22 mg 1.2 equivalents) and DIEA (34 mg, 3 equivalents) in THF (3 ml) at 0° C. The reaction mixture was allowed to warm to room temperature and stirred for 16 hours. A solution of 5,6-dichloroisoindoline (21 mg, 1.2 eq) and TEA (3 eq) in THF (5 ml) was added. The reaction mixture was stirred at 60° C. for 16 hours. After cooled down to room temperature and the mixture was then concentrated under vacuum. The residue was purified by prep-HPLC to give 1.

MS m/z (ESI): 383 [M+1].

¹H NMR (400 MHz, DMSO-d₆) δ 10.47 (s, 1H), 7.55 (s, 2H), 7.32 (d, 1H), 6.23 (t, 1H), 4.52-4.39 (m, 4H), 3.43 (dd, 1H), 3.33 (dd, 1H), 0.99 (ddd, 1H), 0.36-0.25 (m, 2H), 0.21 (dt, 1H).

### (S)-5,6-dichloro-N-((4-cyclopropyl-2,5-dioxoimidazolidin-4-yl)methyl)isoindoline-2-carboxamide 1-1

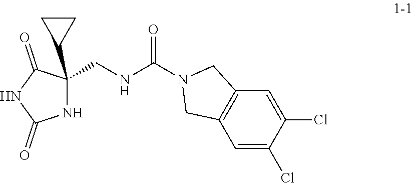 [SMILES: O=C1NC(=O)[C@@](CNC(=O)N2Cc3cc(Cl)c(Cl)cc3C2)(C2CC2)N1]

The title compound was prepared from Int-1A with the similar procedures to Example 1.

MS m/z (ESI): 383 [M+1]

¹H NMR (400 MHz, Methanol-d₄) δ 7.50 (s, 2H), 4.71-4.59 (m, 4H), 3.69 (d, 2H), 1.25 (tt, 1H), 0.67-0.55 (m, 1H), 0.53-0.30 (m, 3H).

### (R)-5,6-dichloro-N-((4-cyclopropyl-2,5-dioxoimidazolidin-4-yl)methyl)isoindoline-2-carboxamide 1-2

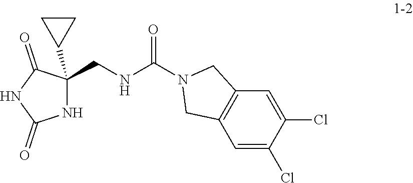 [SMILES: O=C1NC(=O)[C@](CNC(=O)N2Cc3cc(Cl)c(Cl)cc3C2)(C2CC2)N1]

A solution of compound Int-1B (105 mg) in THF (3 ml) was added over 15 mins to a stirring solution of 4-nitrophenyl chloroformate (154 mg 1.2 equivalents) and DIEA (238 mg, 3 equivalents) in THF (10 ml) at 0° C. The reaction mixture was warmed to room temperature and stirred for 16 hours. A solution of 5,6-dichloroisoindoline (1.47 mg, 1.2 eq) and TEA (3 eq) in THF (5 ml) was added. The reaction mixture was stirred at 60° C. for 16 hours. After cooled down to room temperature and the mixture was then concentrated under vacuum. The residue was purified by prep-HPLC to give 1-2 (98 mg, yield 42%).

MS m/z (ESI): 383 [M+H]

¹H NMR (400 MHz, Methanol-d₄) δ 7.52 (s, 2H), 4.66 (t, 4H), 3.69 (d, 2H), 1.25 (tt, 1H) 0.60 (td, 1H), 0.46 (ddt, 2H), 0.42-0.30 (m, 1H).

### Example 2

### 5,6-dichloro-N—(((R)-4-cyclopropyl-2,5-dioxoimidazolidin-4-yl)methyl)-1-methylisoindoline-2-carboxamide 2

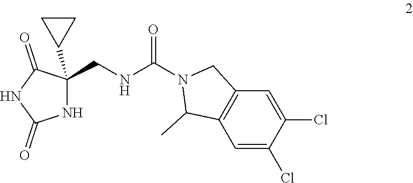 [SMILES: CC1c2cc(Cl)c(Cl)cc2CN1C(=O)NC[C@@]1(C2CC2)NC(=O)NC1=O]

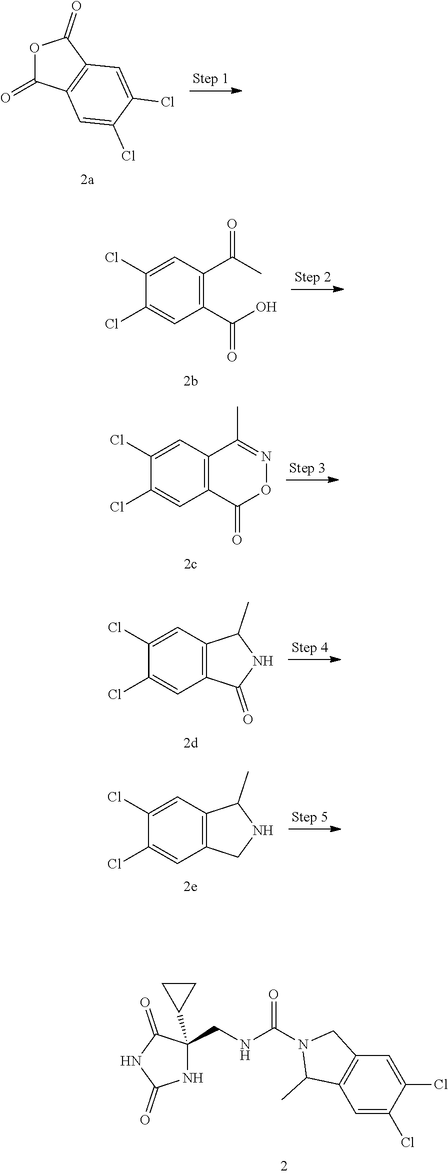 [SMILES: O=C1OC(=O)c2cc(Cl)c(Cl)cc21 ; CC(=O)c1cc(Cl)c(Cl)cc1C(=O)O ; Cc1noc(=O)c2cc(Cl)c(Cl)cc12 ; CC1NC(=O)c2cc(Cl)c(Cl)cc21 ; CC1NCc2cc(Cl)c(Cl)cc21 ; CC1c2cc(Cl)c(Cl)cc2CN1C(=O)NC[C@@]1(C2CC2)NC(=O)NC1=O]

### Step 1

### 2-acetyl-4,5-dichlorobenzoic acid 2b

To a mixture of 5,6-dichloroisobenzofuran-1,3-dione 2a (5 g, 23.04 mmol) and 3,3-dihydroxypropanoic acid (3.67 g, 34.56 mmol) in pyridine (5 mL) was stirred at 75° C. for 2 h. Water (16 mL) and conc. HCl (16 mL) were added, the reaction was stirred at 130° C. for 30 min. The mixture was cooled to r.t, then filtered to give 2b (2.1 g, 39.11% yield).

LCMS: MS m/z (ESI): 230.9 [M−H]⁻.

### Step 2

### 6,7-dichloro-4-methyl-1H-benzo[d][1,2]oxazin-1-one 2c

To a solution of 2b (2.1 g, 9.01 mmol) in water (12 mL) was added KOH (1.52 g, 27.03 mmol). Then hydroxylamine hydrochloride (1.25 g, 18.02 mmol) was slowly added to the solution. The reaction was stirred at r.t for 18 h. The solution was cooled to 0° C., the resulting precipitate was filtered, the solid was dried to give 2c (1.2 g, 57.89% yield).

LCMS: MS m/z (ESI): 230.1 [M+H]⁺.

### Step 3

### 5,6-dichloro-3-methylisoindolin-1-one 2d

To a solution of 2c (3 g, 13.04 mmol) in acetic acid (20 mL) was added Zn (10 g, 153.85 mmol). The reaction was stirred at 115° C. for 24 h. The mixture was cooled to r.t and filtered, the cake was washed with DCM, and the filtrate was concentrated. The residue was purified by SGC (hexane:EtOAc=4:1) to give 2d (2 g, 70.98% yield).

LCMS: MS m/z (ESI): 216.1 [M+H]⁺.

### Step 4

### 5,6-dichloro-1-methylisoindoline 2e

To a solution of 2d (2 g, 9.26 mmol) in THF (10 mL) was added borane-tetrahydrofuran complex (629.44 mg, 37.03 mmol, 20 mL). The reaction was stirred at 60° C. for 18 h. MeOH (2 mL) was added dropwise and HCl (6 M, 2 mL) was added, the reaction was stirred at 80° C. for 2 h. Then NaOH (5 M) was added to adjust the mixture to pH=7, the solution was dried and concentrated. The residue was purified by silica gel chromatography (DCM:MeOH=20:1) to give 2e (900 mg, 4.45 mmol, 48.11% yield).

¹H NMR (400 MHz, DMSO-d₆): δ 7.62 (s, 2H), 4.63-4.58 (m, 1H), 4.31-4.19 (m, 2H), 1.46 (d, 3H).

LCMS: m/z (ESI): 202.1 [M+H]⁺

### Step 5

### 5,6-dichloro-N—(((R)-4-cyclopropyl-5-oxoimidazolidin-4-yl)methyl)-1-methylisoindoline-2-carboxamide 2

To a solution of Int-1B and 4-nitrophenyl chloroformate (745 mg, 3.71 mmol) in DCM (20 mL) was added DIEA (1.14 g, 8.85 mmol). The reaction was stirred at r.t for 18 h. Then 2e (499.33 mg, 2.47 mmol) was added followed by DIEA (958.04 mg, 7.41 mmol) and DMSO (10 mL). The reaction was stirred at 50° C. for 2 h. The mixture was cooled to room temperature. Water (50 mL) was added, and the mixture was extracted with DCM (50 mL×2), the organic solution was washed with brine, dried over Na₂SO₄ and concentrated. The residue was purified by silica gel chromatography (DCM:MeOH=50:1) to give 2 (430 mg, 43.81% yield).

### (R)-5,6-dichloro-N—(((R)-4-cyclopropyl-2,5-dioxoimidazolidin-4-yl)methyl)-1-methylisoindoline-2-carboxamide 2-1

### (S)-5,6-dichloro-N—(((R)-4-cyclopropyl-2,5-dioxoimidazolidin-4-yl)methyl)-1-methylisoindoline-2-carboxamide 2-2

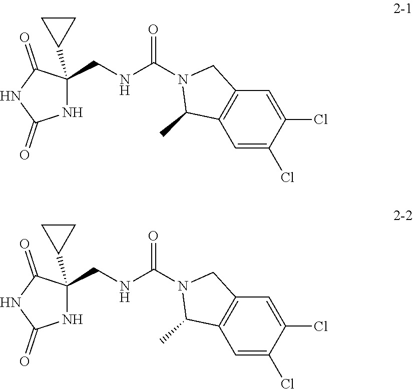 [SMILES: C[C@@H]1c2cc(Cl)c(Cl)cc2CN1C(=O)NC[C@@]1(C2CC2)NC(=O)NC1=O ; C[C@H]1c2cc(Cl)c(Cl)cc2CN1C(=O)NC[C@@]1(C2CC2)NC(=O)NC1=O]

2 (430 mg) was separated by SFC to give title compounds 2-1 (127 mg) and 2-2 (178 mg).

### Compound 2-1

¹H NMR (400 MHz, DMSO-d₆): δ 10.55 (s, 1H), 7.66 (d, 2H), 7.42 (s, 1H), 6.23 (t, 1H), 5.09-5.04 (m, 1H), 4.56 (s, 2H), 3.64-3.59 (m, 1H), 3.38-3.35 (m, 1H), 1.36 (d, 3H), 1.13-1.07 (m, 1H), 0.45-0.26 (m, 3H), 0.12-0.06 (m, 1H).

LCMS: m/z (ESI): 397.1 [M+H]⁺

Chiral HPLC (CO₂/MeOH/DEA 5-40% 1.5 ml/min OJ, 3 um, 3*100 (Daicel)): Rt: 3.041 min, ee: 100%.

### Compound 2-2

¹H NMR (400 MHz, DMSO-d₆): δ 10.59 (s, 1H), 7.66 (d, 2H), 7.40 (d, 1H), 6.38 (t, 1H), 5.12-5.05 (m, 1H), 4.61-4.49 (m, 2H), 3.70 (d, 1H), 3.27 (d, 1H), 1.36 (d, 3H), 1.12-1.05 (m, 1H), 0.44-0.27 (m, 3H), 0.15-0.07 (m, 1H).

LCMS: m/z (ESI): 397.1 [M+14]⁺

Chiral HPLC (CO₂/MeOH/DEA 5%-40% 1.5 ml/min OJ, 3 um, 3*100 (Daicel)): Rt: 2.714 min, ee: 100%.

### Example 3

### (R)-5-chloro-N-((4-cyclopropyl-2,5-dioxoimidazolidin-4-yl)methyl)-6-(trifluoromethyl)isoindoline-2-carboxamide 3

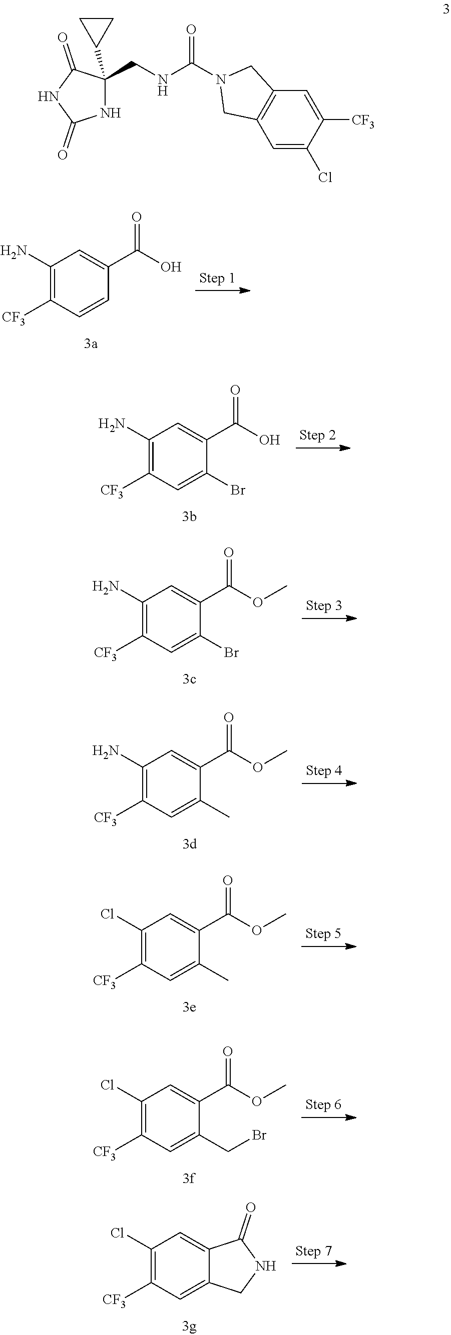

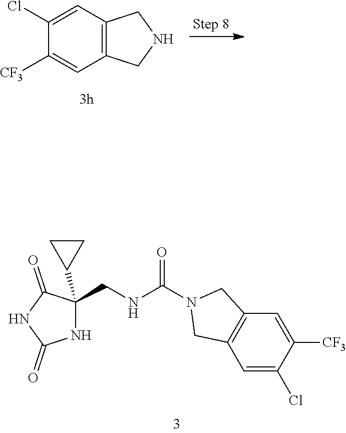 [SMILES: FC(F)(F)c1cc2c(cc1Cl)CNC2 ; O=C1NC(=O)[C@](CNC(=O)N2Cc3cc(Cl)c(C(F)(F)F)cc3C2)(C2CC2)N1]

### Step 1

### 5-amino-2-bromo-4-(trifluoromethyl)benzoic acid 3b

To a solution of 3-amino-4-(trifluoromethyl)benzoic acid 3a (1 g, 4.87 mmol) in DMF (20 mL) was added NBS (870 mg, 4.89 mmol). The mixture was stirred at room temperature for 2 hours, the resulting mixture was poured into ice water (20 mL) and the mixture was extracted with EtOAc (20 mL×2). The combined organic phase was washed with water (20 mL), brine (20 mL), dried over Na₂SO₄ and filtered. The filtrate was concentrated to afford crude 3b (1 g, 3.52 mmol, 72.2% yield).

### Step 2

### Methyl 5-amino-2-bromo-4-(trifluoromethyl)benzoate 3e

To a solution of 3b (1 g, 3.52 mmol) in MeOH (10 mL) was added H₂SO₄ (18 M, 0.7 mL) dropwise. After the mixture was stirred at 75° C. overnight, the mixture was cooled down to room temperature and poured into ice water (20 mL), the mixture was extracted with EtOAc (50 mL). The organic phases were dried over Na₂SO₄ and filtered. The filtrate was concentrated to afford crude 3e (1 g, 3.36 mmol, 95.3% yield).

¹H NMR (400 MHz, DMSO-d₆): δ 7.57 (s, 1H), 7.21 (s, 1H), 6.11 (brs, 2H), 3.85 (s, 3H).

### Step 3

### Methyl 5-amino-2-methyl-4-(trifluoromethyl)benzoate 3d

To a solution of 3e (1 g, 3.36 mmol) in DMF (10 mL) was added Pd(PPh₃)₄ (430 mg, 372.11 umol), K₃PO₄ (2.2 g, 10.36 mmol) and methylboronic acid (1 g, 16.71 mmol). After the mixture was stirred at 130° C. under N₂ atmosphere overnight, the mixture was cooled down to room temperature and filtered. The filtrate was concentrated, and the residue was purified by silica gel chromatography to afford 3d (500 mg, 2.14 mmol, 63.9% yield).

LCMS: MS m/z (ESI); 234.1 [M+H]⁺

### Step 4

### Methyl 5-chloro-2-methyl-4-(trifluoromethyl)benzoate 3e

Concentrated HCl (2 mL) was added to a solution of 3d (2.0 g, 8.58 mmol) in acetone (20 mL), and the mixture was stirred at RT for 20 min. Then the mixture was cooled to −5 to 0° C. Then a solution of NaNO₂ (600 mg, 8.70 mmol) in H₂O (2.5 mL) was added dropwise, and the mixture was stirred at an ambient temperature for 30 min. CuCl (849.11 mg, 8.58 mmol) was added portion-wise at 0° C., and the mixture was stirred at RT for 2 h. After the completion of the reaction, the mixture was poured into 1N HCl (50 mL) and the mixture was extracted with EtOAc. The combined organic layer was washed with water and brine, dried over anhydrous Na₂SO₄, filtered and concentrated in vacuo. The residue was purified by column chromatography to afford 3e (1.3 g, 5.15 mmol, 60% yield).

### Step 5

### Methyl 2-bromo-5-chloro-4-(trifluoromethyl)benzoate 3f

To a solution of 3e (1.3 g, 5.15 mmol) in CCl₄ (20 mL) was added NBS (1.10 g, 6.18 mmol) and AIBN (25.35 mg, 154.38 umol), the mixture was heated to 70° C. and stirred overnight. The mixture was cooled to RT and filtered, the cake was washed with CCl₄, the filtrate was concentrated in vacuo to give crude 3f which is used directly to next step without further purification.

### Step 6

### 6-chloro-5-(trifluoromethyl)isoindolin-1-one 3g

To a solution of 3f (1.9 g, 5.73 mmol) in MeOH (10 mL) was added NH₃/MeOH (20 mL) and the mixture was stirred at RT overnight. The reaction mixture was concentrated in vacuo. The residue was purified by column chromatography to afford 3g (920 mg, 3.91 mmol, 68.1% yield). LCMS: MS m/z (ESI); 236.0 [M+H]⁺

### Step 7

### 5-chloro-6-(trifluoromethyl)isoindoline 3h

To a solution of 3g (440 mg, 1.87 mmol) in THF (5 mL) was added BH₃/THF (1N, 20 mL). The mixture was heated to 60° C. overnight. The reaction solution was quenched with methanol (5 mL) and 6 M HCl was added to adjusted pH to 1-2. Then the mixture was heated to 80° C. and stirred for 1 h. The reaction was cooled to RT and the mixture was adjusted pH to 7-8 with 6N aq. NaOH. The mixture was directly dried over anhydrous Na₂SO₄, filtered and concentrated in vacuo. The crude was purified by column chromatography to afford 3h (240 mg, 1.08 mmol, 58% yield).

LCMS; MS m/z (ESI): 222.3 [M+H]⁺

¹H NMR (400 MHz, DMSO-d₆): δ 7.78 (s, 1H), 7.65 (s, 1H), 4.16 (br, 2H), 4.14 (br, 2H).

### Step 8

### (R)-5-chloro-N-((4-cyclopropyl-2,5-dioxoimidazolidin-4-yl)methyl)-6-(trifluoromethyl)isoindoline-2-carboxamide 3

The solution of the 4-nitrophenyl chloroformate (142.97 mg, 709.30 umol) in DCM (5 mL) was stirred at 0° C. 20 min. Then a mixture of Int-1B (80 mg, 472.87 umol), DIEA (305.57 mg, 2.36 mmol) in DCM (5 mL) was added dropwise at 0° C. The reaction was stirred at RT overnight. Then a solution of 5-chloro-6-(trifluoromethyl)isoindoline 3h (110 mg, 496.38 umol), TEA (143.55 mg, 1.42 mmol) in DMSO (5 mL) was added. The reaction was heated to 50° C. and stirred for 4 h. Water was added, and the mixture was extracted with DCM. The combined organic layers were washed with water and brine, dried over anhydrous Na₂SO₄, filtered and concentrated in vacuo. The residue was purified by prep-HPLC to give 3 (19.6 mg, 47.03 umol, 9.95% yield).

¹HNMR (400 MHz, DMSO-d₆): δ 10.59 (brs, 1H), 7.88 (brs, 1H), 7.76 (brs, 1H), 7.45 (brs, 1H), 6.43-6.34 (m, 1H), 4.66 (br, 2H), 4.64 (br, 2H), 3.57-3.45 (m, 2H), 1.15-1.05 (m, 1H), 0.49-0.27 (m, 3H), 0.10 (br, 1H).

¹⁹F NMR (376.5 MHz, DMSO-d₆): δ −60.79.

LCMS: MS m/z (ESI); 417.3 [M+H]⁺,

### Example 4

### N-((4-cyclopropyl-2,5-dioxoimidazolidin-4-yl)methyl)-5-(trifluoromethyl)isoindoline-2-carboxamide

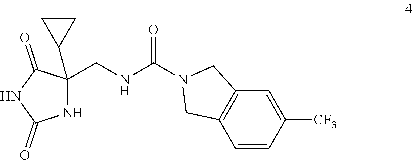 [SMILES: O=C1NC(=O)C(CNC(=O)N2Cc3ccc(C(F)(F)F)cc3C2)(C2CC2)N1]

A solution of Int-1 (126 mg, 0.74 mmol) and DIEA (239 mg, 1.85 mmol) in DCM (3 mL) was added to a mixture of 4-nitrophenyl chloroformate (223 mg, 1.11 mmol) in DCM (3 mL) at 0° C. The reaction mixture was stirred at r.t for 18 h. A solution of 5-(trifluoromethyl)isoindoline (164 mg, 0.74 mmol) and TEA (187 mg, 1.85 mmol) in DMSO (3 mL) was added. The reaction mixture was stirred at 50° C. for 18 h. Water (50 mL) was added, and the mixture was extracted with EtOAc (50 mL×2), the organic solution was washed with brine, dried over Na₂SO₄ and concentrated. The residue was purified by prep-HPLC to give 4 (150 mg, 0.39 mmol, 52.82% yield).

¹H NMR (400 MHz, DMSO-d₆): δ 10.60 (s, 1H), 7.75 (s, 1H), 7.66 (d, 1H), 7.56 (d, 1H), 7.46 (s, 1H), 6.39-6.35 (t, 1H), 4.71-4.62 (m, 4H), 3.59-3.53 (m, 1H), 3.48-3.34 (m, 1H), 1.15-1.07 (m, 1H), 0.46-0.27 (m, 3H), 0.14-0.07 (m, 1H).

LCMS: MS m/z (ESI): 383.4 [M+H]⁺.

### Example 5

### N-((4-cyclopropyl-2,5-dioxoimidazolidin-4-yl)methyl)-N-methyl-5-(trifluoromethyl)isoindoline-2-carboxamide 5

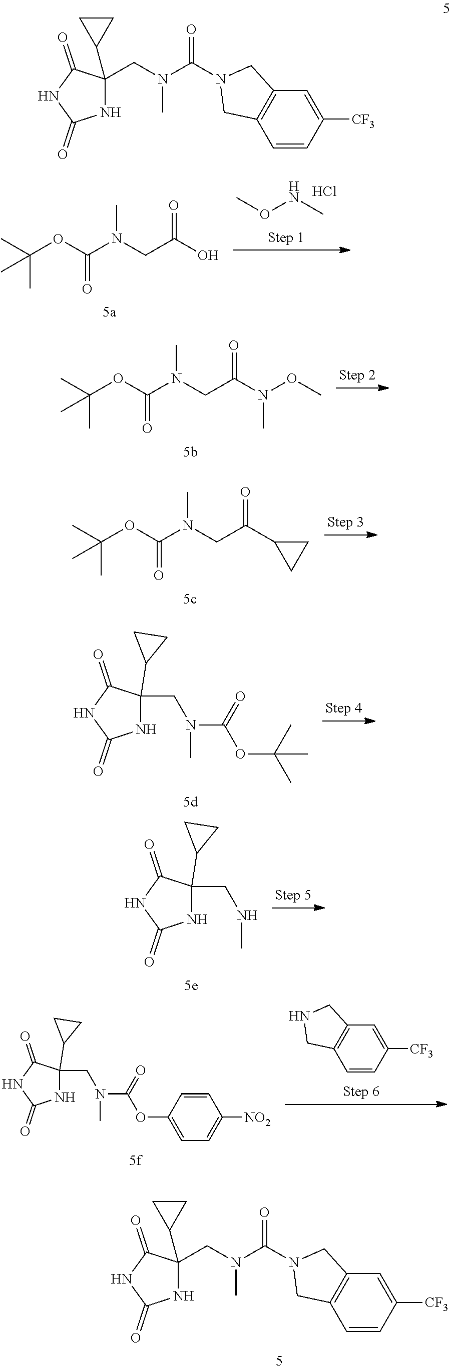

### Tert-butyl N-[2-[methoxy(methyl)amino]-2-oxo-ethyl]-N-methyl-carbamate 5b

To a solution of 2-((tert-butoxycarbonyl)(methyl)amino)acetic acid 5a (5 g, 26.43 mmol) in DCM (80 mL) was added TEA (8.82 g, 87.21 mmol). The resulting mixture was stirred at 0-5° C. for 20 min, followed by the addition of HOBt (3.57 g, 26.43 mmol) and EDCI (6.1 g, 31.71 mmol). The resulting mixture was stirred at 0-5° C. for 30 min, followed by the addition of N, O-dimethylhydroxylamine HCl salt (2.84 g, 29.07 mmol). The resulting mixture was stirred at RT for 16 h and the TLC indicated the reaction was completed. The mixture was poured into water (150 mL), the aqueous phase was extracted with EtOAc (200 mL*3), the combined organic phase was washed with brine (200 mL), dried over Na₂SO₄, filtered and concentrated under reduced pressure. The residue was stirred in hexane at rt for 30 min. The solid was collected by filtration and further dried in vacuo to afford 5b (4.9 g, 21.10 mmol, 79.8% yield).

¹H NMR (400 MHz, CDCl₃): δ 4.16-4.07 (m, 2H), 3.71 (d, 3H), 3.19 (s, 3H), 2.93 (s, 3H), 1.46 (d, 9H).

### Step 2

### Tert-butyl N-(2-cyclopropyl-2-oxo-ethyl)-N-methyl-carbamate 5c

To a solution of 5b (4.94 g, 21.26 mmol) in THF (10 mL) was added bromo(cyclopropyl)magnesium (9.27 g, 63.8 mmol, 65 mL) dropwise at 10° C. The resulting mixture was stirred at 10° C. for 4 h. The reaction was quenched with HCl, the aqueous phase was extracted with EtOAc (150 mL×3), the combined organic phase was washed with brine (200 mL), dried over Na₂SO₄, filtered and concentrated under reduced pressure. The residue was purified by silica gel column chromatography to afford 5c (1.1 g, 5.16 mmol, 24.3% yield).

¹H NMR (400 MHz, CDCl₃): δ 4.11 (d, 2H), 2.96 (d, 3H), 1.94-1.92 (m, 1H), 1.47 (d, 9H), 1.09-1.05 (m, 2H), 0.92 (br, 2H).

### Step 3

### Tert-butyl N-[(4-cyclopropyl-2,5-dioxo-imidazolidin-4-yl)methyl]-N-methyl-carbamate 5d

To a solution of 5c (1.1 g, 5.16 mmol) in MeOH (10 mL) and water (10 mL) was added (NH₄)₂CO₃ (2.67 g, 27.85 mmol) and NaCN (683.91 mg, 12.89 mmol), The resulting mixture was stirred at 80° C. in a sealed tube for 16 h. The mixture was poured into water (200 mL), the aqueous phase was extracted with EtOAc (100 mL×3), the combined organic phase was washed with brine (200 mL), dried over Na₂SO₄, filtered and concentrated under reduced pressure. The residue was recrystallized with hexane/EtOAc to afford 5d (1 g, 3.53 mmol, 68.43% yield). ¹H NMR (400 MHz, CDCl₃): δ 8.07 (brs, 1H), 5.83 (brs, 1H), 3.99 (d, 1H), 3.38 (d, 1H), 2.88 (s, 3H), 1.45 (s, 9H), 1.13 (br, 1H), 0.67-0.54 (m, 1H), 0.47-0.36 (m, 3H).

### Step 4

### 5-cyclopropyl-5-(methylaminomethyl)imidazolidine-2,4-dione 5e

To a solution of 5d (100 mg, 352.95 umol) in MeOH (1 mL) was added HCl/dioxane (4N, 1.5 mL, 6.0 mmol). The resulting mixture was stirred at RT for 2 h. The reaction mixture was concentrated to afford crude 5e (70 mg, 318.66 umol, 90.3% yield).

### Step 5

### 4-nitrophenyl ((4-cyclopropyl-2,5-dioxoimidazolidin-4-yl)methyl)(methyl)carbamate 5f

To a solution of 5e (70 mg, 469 umol) in DCM (4 mL) was added DIEA (181.46 mg, 1.41 mmol) and 4-nitrophenyl chloroformate (95 mg, 469 umol). The resulting mixture was stirred at RT for 16 h and the TLC indicated the reaction was finished. The mixture used without purification.

### Step 6

### N-((4-cyclopropyl-2,5-dioxoimidazolidin-4-yl)methyl)-N-methyl-5-(trifluoromethyl)isoindoline-2-carboxamide 5

To a solution of 5f (from previous step) were added DMSO (3 mL), 5-(trifluoromethyl)isoindoline (110 mg, 468.89 umol) and TEA (42.41 mg, 419.10 umol). The resulting mixture was stirred at 50° C. for 16 h. The mixture was purified by prep-HPLC to afford (4 mg, 10.1 umol, 2.4% yield).

¹H NMR (400 MHz, DMSO-d₆): δ 10.64 (brs, 1H), 7.70 (d, 2H), 7.65 (d, 1H), 7.54 (d, 1H), 4.80-4.66 (m, 4H), 3.75-3.65 (m, 2H), 2.98 (s, 3H), 1.10-1.05 (m, 1H), 0.45-0.27 (m, 3H), 0.20-0.05 (m, 1H).

¹⁹F NMR (376.5 MHz, DMSO-d₆): δ −60.51

LCMS: MS m/z (ESI): 397.1 [M+H]⁺

### Example 8

### 5-chloro-N—(((R)-4-cyclopropyl-2,5-dioxoimidazolidin-4-yl)methyl)-1-methyl-6-(trifluoromethyl)isoindoline-2-carboxamide 8

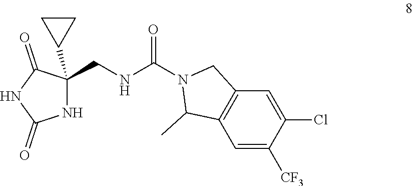 [SMILES: CC1c2cc(C(F)(F)F)c(Cl)cc2CN1C(=O)NC[C@@]1(C2CC2)NC(=O)NC1=O]

### Example 8-1

### (R)-5-chloro-N—(((R)-4-cyclopropyl-2,5-dioxoimidazolidin-4-yl)methyl)-1-methyl-6-(trifluoromethyl)isoindoline-2-carboxamide 8-1

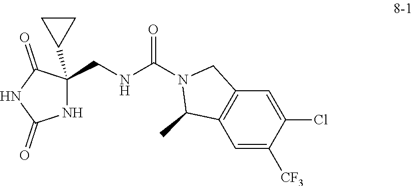 [SMILES: C[C@@H]1c2cc(C(F)(F)F)c(Cl)cc2CN1C(=O)NC[C@@]1(C2CC2)NC(=O)NC1=O]

### Example 8-2

### (S)-5-chloro-N—(((R)-4-cyclopropyl-2,5-dioxoimidazolidin-4-yl)methyl)-1-methyl-6-(trifluoromethyl)isoindoline-2-carboxamide 8-2

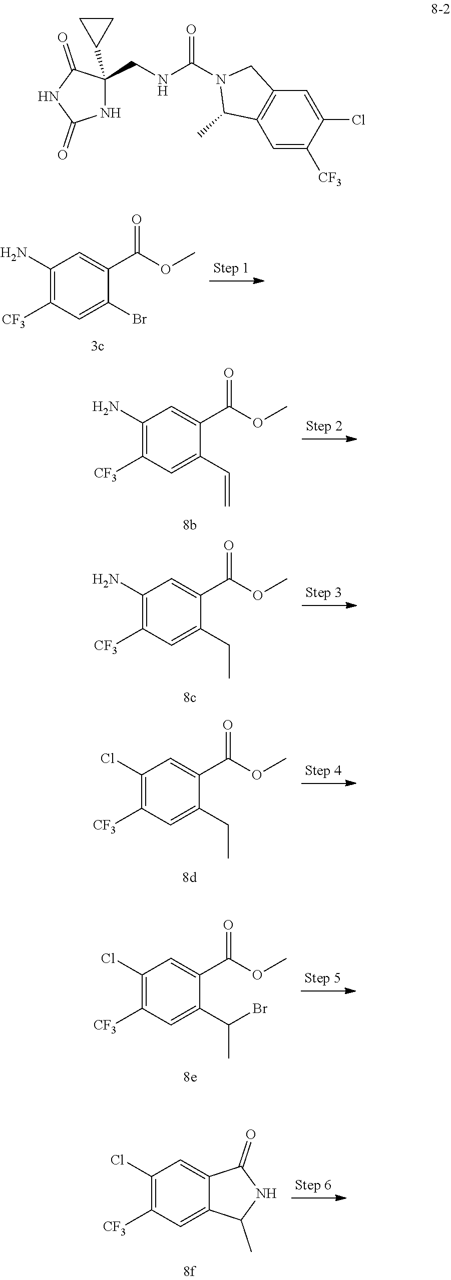

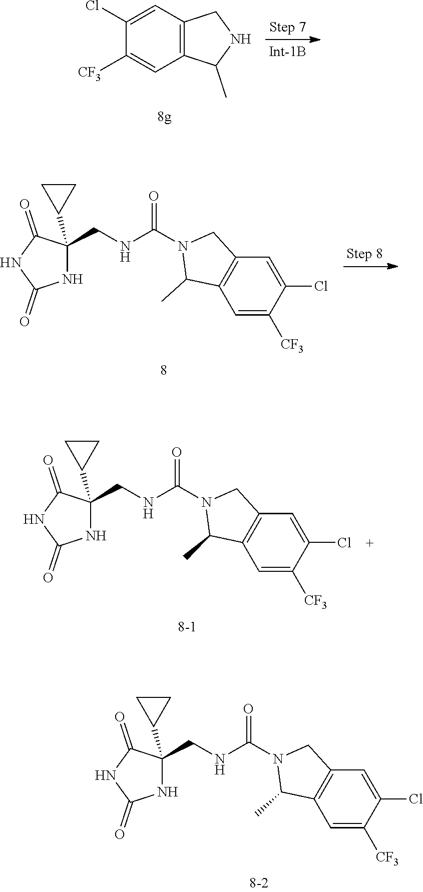 [SMILES: CC1NCc2cc(Cl)c(C(F)(F)F)cc21 ; CC1c2cc(C(F)(F)F)c(Cl)cc2CN1C(=O)NC[C@@]1(C2CC2)NC(=O)NC1=O ; C[C@@H]1c2cc(C(F)(F)F)c(Cl)cc2CN1C(=O)NC[C@@]1(C2CC2)NC(=O)NC1=O ; C[C@H]1c2cc(C(F)(F)F)c(Cl)cc2CN1C(=O)NC[C@@]1(C2CC2)NC(=O)NC1=O]

### Step 1

### Methyl 5-amino-4-(trifluoromethyl)-2-vinyl-benzoate 8b

To a solution of 3c (5.45 g, 18.29 mmol) and potassium vinyltrifluoroborate (2.45 g, 18.29 mmol) in dioxane (50 mL) and water (10 mL) was added Pd(dppf)Cl₂ (1.34 g, 1.83 mmol) and K₂CO₃ (6.35 g, 45.71 mmol). The resulting mixture was evacuated and refilled with N₂ for 3 times. The resulting mixture was stirred at 80° C. for 16 h. The mixture was diluted with EtOAc (100 mL), the combined organic phase was washed with brine (100 mL), dried over Na₂SO₄, filtered and concentrated under reduced pressure. The residue was purified by silica gel column chromatography using EtOAc/hexane as eluent to afford the tittle compound 8b (3.56 g, 14.52 mmol, 79.4% yield).

LCMS: MS m/z (ESI): 246.1 [M+H]⁺

### Step 2

### Methyl 5-amino-2-ethyl-4-(trifluoromethyl) benzoate 8c

To a solution of 8b (3.56 g, 14.52 mmol) in MeOH (20 mL) was added 10% Pd/C (1.55 g, 1.45 mmol). The resulting mixture was evacuated and refilled with H₂. The resulting mixture was stirred at RT for 16 h and the LCMS indicated the reaction was finished. The mixture was filtered, and the cake was washed with MeOH, the filtrate was concentrated under reduced pressure to afford the tittle compound 8e (3.45 g, 13.96 mmol, 96.1% yield).

LCMS: MS m/z (ESI): 248.1 [M+H]⁺

### Step 3

### Methyl 5-chloro-2-ethyl-4-(trifluoromethyl) benzoate 8d

To a solution of 8c (3.36 g, 13.59 mmol) in acetone (34 mL) was added HCl (3.36 mL). The resulting mixture was stirred at RT for 20 min. After the mixture was cooled to 0° C., a solution of NaNO₂ (1.88 g, 27.18 mmol) in water (5 mL) was added. Then CuCl (1.48 g, 14.95 mmol) was added in portions at 0° C. The resulting mixture was stirred at RT for 1 h. The mixture was poured into 1 M HCl (60 mL), the aqueous phase was extracted with EtOAc (100 mL*3), the combined organic phase was washed with brine (100 mL), dried over Na₂SO₄, filtered and concentrated under reduced pressure. The residue was purified by silica gel column chromatography (eluting with hexane/EtOAc=50/l) to afford the title compound 8d (2.23 g, 8.36 mmol, 61.53% yield).

¹H NMR (400 MHz, DMSO-d₆): δ 7.99 (s, 1H), 7.87 (s, 1H), 3.88 (s, 3H), 2.92 (q, 2H), 1.17 (t, 3H).

### Step 4

### Methyl 2-(1-bromoethyl)-5-chloro-4-(trifluoromethyl) benzoate 8e

To a solution of 5d (2.23 g, 8.36 mmol) in CCl₄ (35 mL) was added AIBN (412.00 mg, 2.51 mmol) and NBS (1.64 g, 9.20 mmol). The resulting mixture was stirred at 80° C. for 16 h. The mixture was filtered. The solid was washed with DCM and the filtrate was concentrated in vacuo to afford the crude tittle compound 8e (2.5 g, 7.24 mmol, 86.51% yield).

¹H NMR (400 MHz, DMSO-d₆): δ 8.17 (s, 1H), 8.04 (s, 1H), 6.08 (q, 1H), 3.92 (s, 3H), 2.05 (d, 3H).

### Step 5

### 6-chloro-3-methyl-5-(trifluoromethyl) isoindolin-1-one 8f

To a solution of 8e (2.5 g, 7.24 mmol) in MeOH (10 mL) was added NH₃/MeOH (7 M, 30 mL), The resulting mixture was stirred at RT for 1.6 h. The mixture was purified by prep-HPLC using CH₃CN/H₂O as eluent to afford the tittle compound 8f (1.18 g, 4.73 mmol, 65.3% yield).

¹H NMR (400 MHz, DMSO-d₆): δ 9.11 (brs, 1H), 8.20 (s, 1H), 7.91 (s, 1H), 4.71 (q, 1H), 1.42 (d, 3H).

¹⁹F NMR (376.5 MHz, DMSO-d₆): δ −60.99.

LCMS: MS mix (ESI): 250.0 [M+H]⁺

### Step 6

### 5-chloro-1-methyl-6-(trifluoromethyl) isoindoline 8g

To a solution of 8f (730 mg, 2.92 mmol) in THF (5 mL) was added BH₃/THF (2 M, 30.93 mL). The resulting mixture was stirred at 60° C. for 16 h. The mixture was quenched with MeOH (5 mL) and HCl (4 M, 5 mL), the mixture was stirred at 60° C. for 3 h. The mixture was basified with aq. NaOH and extracted with EtOAc (50 mL×3), the combined organic phase was washed with brine (50 mL), dried over Na₂SO₄, filtered and concentrated under reduced pressure. The residue was purified by silica gel column chromatography (DCM/MeOH=20/1) to afford the tittle compound 8g (300 mg, 1.27 mmol, 43.5% yield).

LCMS: MS m/z (ESI): 236.1 [M+H]⁺.

### Step 7

### 5-chloro-N—(((R)-4-cyclopropyl-2,5-dioxoimidazolidin-4-yl)methyl)-1-methyl-6-(trifluoromethyl)isoindoline-2-carboxamide 8

A solution of compound Int-1B (51 mg, 249 umol) in THF (3 mL) was added over 15 mins to a stirring solution of 4-nitrophenyl chloroformate (60 mg, 298 umol) and DIEA (90 mg, 894 umol) in THF (10 ml) at 0° C. The reaction mixture was warmed to room temperature and stirred for 16 hours. Then a solution of 8g (80.00 mg, 294.02 umol) in TEA (148.76 mg, 1.47 mmol) was added. The resulting mixture was stirred at 50° C. for 2 h. The mixture was purified by prep-HPLC using CH₃CN/H₂O as eluent to afford the tittle compound 8 (70 mg, 162.49 umol, 55.3% yield).

¹H NMR (400 MHz, DMSO-d₆): δ 10.59 (d, 1H), 7.87 (s, 1H), 7.74 (d, 1H), 7.42 (d, 1H), 6.35 (dt, 6.4 Hz, 1H), 5.17-5.13 (m, 1H), 4.69-4.58 (m, 2H), 3.73-3.59 (m, 1H), 3.40-3.25 (m, 1H), 1.42-1.38 (m, 3H), 1.15-1.05 (m, 1H), 0.43-0.29 (m, 3H), 0.13-0.07 (m, 1H).

¹⁹F NMR (376.5 MHz, DMSO-d₆): δ −60.68.

HPLC: 99.8% @214 nm.

LCMS: MS m/z (ESI): 431.0 [M+H]⁺.

### Step 8

### (R)-5-chloro-N—(((R)-4-cyclopropyl-2,5-dioxoimidazolidin-4-yl)methyl)-1-methyl-6-(trifluoromethyl)isoindoline-2-carboxamide 8-1 & (S)-5-chloro-N—(((R)-4-cyclopropyl-2,5-dioxoimidazolidin-4-yl)methyl)-1-methyl-6-(trifluoromethyl)isoindoline-2-carboxamide 8-2

Compound 8 (70 mg, 162.49 umol) was separated by SFC to afford the tittle compounds 8-1 (23 mg) and 8-2 (20 mg).

### Compound 8-1

¹H NMR (400 MHz, DMSO-d₆): δ 10.55 (brs, 1H), 7.87 (s, 1H), 7.75 (s, 1H), 7.42 (s, 1H), 6.27 (t, 1H), 5.15-5.13 (m, 1H), 4.65 (br, 2H), 3.65-3.59 (m, 1H), 3.41-3.37 (m, 1H), 1.40 (d, 3H), 1.13-1.09 (m, 1H), 0.45-0.30 (m, 3H), 0.12-0.09 (m, 1H).

¹⁹F NMR (376.5 MHz, DMSO-d₆): 5-60.68.

HPLC: 99.3% @ 214 nm.

LCMS: MS m/z (ESI): 431.0 [M+H]⁺.

Chiral HPLC (CO₂/MeOH/DEA 5%-40% 1.5 ml/min OJ, 3 um, 3*100 (Daicel)): Rt: 0.813 min, ee: 100%.

### Compound 8-2

¹H NMR (400 MHz, DMSO-d₆): δ 10.55 (br, 1H), 7.90-7.86 (m, 1H), 7.78-7.73 (m, 1H), 7.42-7.39 (m, 1H), 6.41 (t, 1H), 5.17-5.14 (m, 1H), 4.69-4.62 (m, 2H), 3.73-3.69 (m, 1H), 3.36-3.30 (m, 1H), 1.44-1.36 (m, 3H), 1.10-1.06 (m, 1H), 0.46-0.37 (m, 3H), 0.12-0.09 (m, 1H).

¹⁹F NMR (376.5 MHz, DMSO-d₆): δ −60.68.

HPLC: 99.3% @ 214 nm.

LCMS: MS m/z (ESI): 431.0 [M+H]⁺.

Chiral HPLC (CQ₂/MeOH/DEA 5%-40% 1.5 ml/min OJ, 3 um, 3*100 (Daicel)): Rt: 1.615 min, ee: 100%.

### Example 9

### 5-chloro-N—(((R)-4-cyclopropyl-2,5-dioxoimidazolidin-4-yl)methyl)-1-methyl-6-(trifluoromethyl)isoindoline-3,3-d₂-2-carboxamide 9

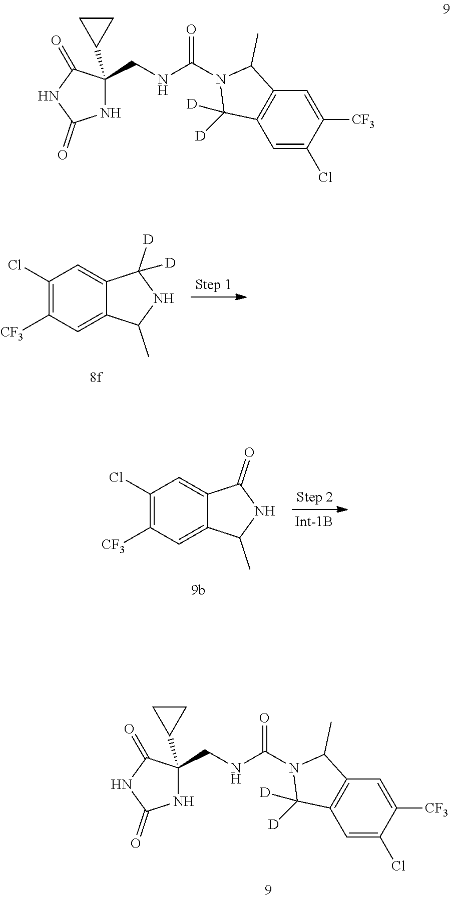 [SMILES: [2H]C1([2H])c2cc(Cl)c(C(F)(F)F)cc2C(C)N1C(=O)NC[C@@]1(C2CC2)NC(=O)NC1=O ; [2H]C1([2H])NC(C)c2cc(C(F)(F)F)c(Cl)cc21 ; CC1NC(=O)c2cc(Cl)c(C(F)(F)F)cc21 ; [2H]C1([2H])c2cc(Cl)c(C(F)(F)F)cc2C(C)N1C(=O)NC[C@@]1(C2CC2)NC(=O)NC1=O]

### Step 1

### 5-chloro-1-methyl-6-(trifluoromethyl)isoindoline-3,3-d₂ 9b

To a solution of 6-chloro-3-methyl-5-(trifluoromethyl)isoindolin-1-one 8f (50 mg, 0.2 mmol) in THF (2 mL) was added Borane-d₃-THF complex solution (1.0 M, 6 mL, 6 mmol). The reaction was stirred at 60° C. for 18 h. MeOH (2 mL) was added dropwise and followed by HCl (6 M, 2 mL), the reaction mixture was stirred at 80° C. for additional 2 h. Then NaOH (5 M) was added to adjust the mixture to pH=7. The reaction mixture was worked up by extraction and the organic layer was dried, filtered and concentrated. The residue was purified by silica gel chromatography (DCM:MeOH=20:1) to give the title compound 9b (39 mg, 0.147 mmol, 70% yield).

### Step 2

### 5-chloro-N—(((R)-4-cyclopropyl-2,5-dioxoimidazolidin-4-yl)methyl)-1-methyl-6-(trifluoromethyl)isoindoline-3,3-d₂-2-carboxamide 9

The solution of the 4-nitrophenyl chloroformate (22 mg, 120 umol) in DCM (5 mL) was stirred at 0° C. for 20 min. Then a mixture of Int-1B (19 mg, 115 umol), DIEA (24 mg, 0.236 mmol) in DCM (5 mL) was added dropwise at 0° C. The reaction was stirred at RT overnight. Then a solution of 9b (18 mg, 89.38 umol), TEA (39.55 mg, 0.3 mmol) in DMSO (5 mL) was added. The reaction was heated to 60° C. and stirred for 4 h. Water was added, and the mixture was extracted with DCM. The combined organic layers were washed with water and brine, dried over anhydrous Na₂SO₄, filtered and concentrated in vacuo. The residue was purified by prep-HPLC to give the title compound 9 (16 mg, 35% yield).

1H NMR (400 MHz, Methanol-A): δ 7.73 (d, 1H), 7.59 (d, 1H), 5.27-5.18 (m, 1H), 3.83 (dd, 1H), 3.56 (dd, 1H), 1.50 (t, 3H), 1.31-1.20 (m, 1H), 0.67-0.55 (m, 2H), 0.52-0.33 (m, 2H).

LCMS: MS m/z (ESI): 433.0 [M+H]⁺.

### Example 11

### 5,6-dichloro-N—(((R)-4-cyclopropyl-2,5-dioxoimidazolidin-4-yl)methyl)-1-methylisoindoline-3,3-d₂-2-carboxamide 11

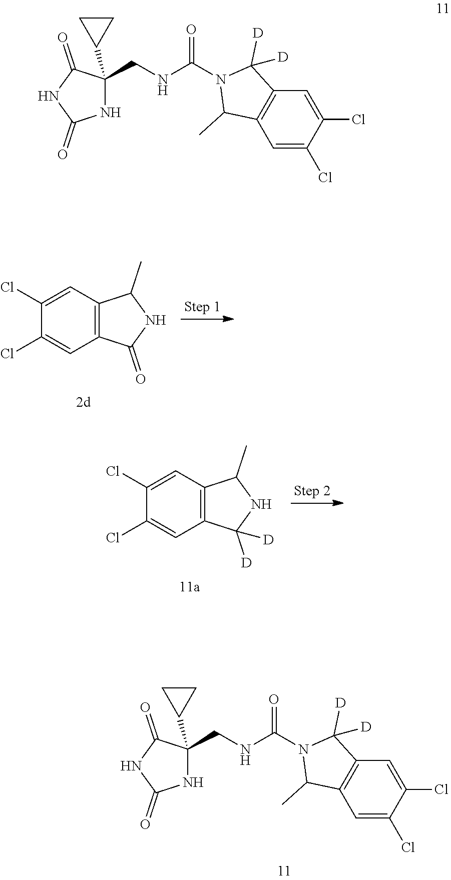 [SMILES: [2H]C1([2H])c2cc(Cl)c(Cl)cc2C(C)N1C(=O)NC[C@@]1(C2CC2)NC(=O)NC1=O ; CC1NC(=O)c2cc(Cl)c(Cl)cc21 ; [2H]C1([2H])NC(C)c2cc(Cl)c(Cl)cc21 ; [2H]C1([2H])c2cc(Cl)c(Cl)cc2C(C)N1C(=O)NC[C@@]1(C2CC2)NC(=O)NC1=O]

### Step 1

### 5,6-dichloro-1-methylisoindoline-3,3-d₂ 11a

To a solution of 2d (100 mg, 0.5 mmol) in THF (5 mL) was added BD₃/THF (1N, 15 mL). The mixture was heated to 60° C. overnight. The reaction solution was quenched with methanol (5 mL) and 6 M HCl was added to adjusted pH to 1-2. Then the mixture was heated to 80° C. and stirred for 1 h. The reaction was cooled to RT and the mixture was adjusted pH to 7-8 with 6 M NaOH, The mixture was directly dried over anhydrous Na₂SO₄ and concentrated in vacuo. The crude was purified by column chromatography to afford 5,6-dichloro-1-methylisoindoline-3,3-d₂ (78 mg, 80% yield).

### Step 2

### 5,6-dichloro-N—(((R)-4-cyclopropyl-2,5-dioxoimidazolidin-4-yl)methyl)-1-methylisoindoline-3,3-d₂-2-carboxamide 11

The solution of the 4-nitrophenyl chloroformate (142.97 mg, 709.30 umol) in DCM (5 mL) was stirred at 0° C. for 20 min. Then a mixture of Int-1B (80 mg, 472.87 umol), DIEA (305.57 mg, 2.36 mmol) in DCM (5 mL) was added dropwise at 0° C. The reaction was stirred at RT overnight. Then a solution of 5,6-dichloro-1-methylisoindoline-3,3-d₂ (110 mg, 496.38 umol), TEA (143.55 mg, 1.42 mmol) in DMSO (5 mL) was added. The reaction was heated to 50° C. and stirred for 4 h. Water was added, and the mixture was extracted with DCM. The combined organic layers were washed with water and brine, dried over anhydrous Na₂SO₄, filtered and concentrated in vacuo. The residue was purified by prep-HPLC to give 11 (26 mg, 16% yield).

1H NMR (400 MHz, Methanol-d₄): δ 7.49 (d, 2H), 5.15 (dq, 1H), 3.82 (dd, 1H), 3.56 (dd, 1H), 1.47 (t, 3H), 1.43-1.18 (m, 1H), 0.53-0.42 (m, 2H), 0.46-0.30 (m, 2H).

LCMS: MS m/z (ESI): 399 [M+H]⁺.

### Example 12

### (R)—N-((4-cyclopropyl-2,5-dioxoimidazolidin-4-yl)methyl)-5-(difluoromethyl)-6-(trifluromethyl)isoindoline-2-carboxamide 12

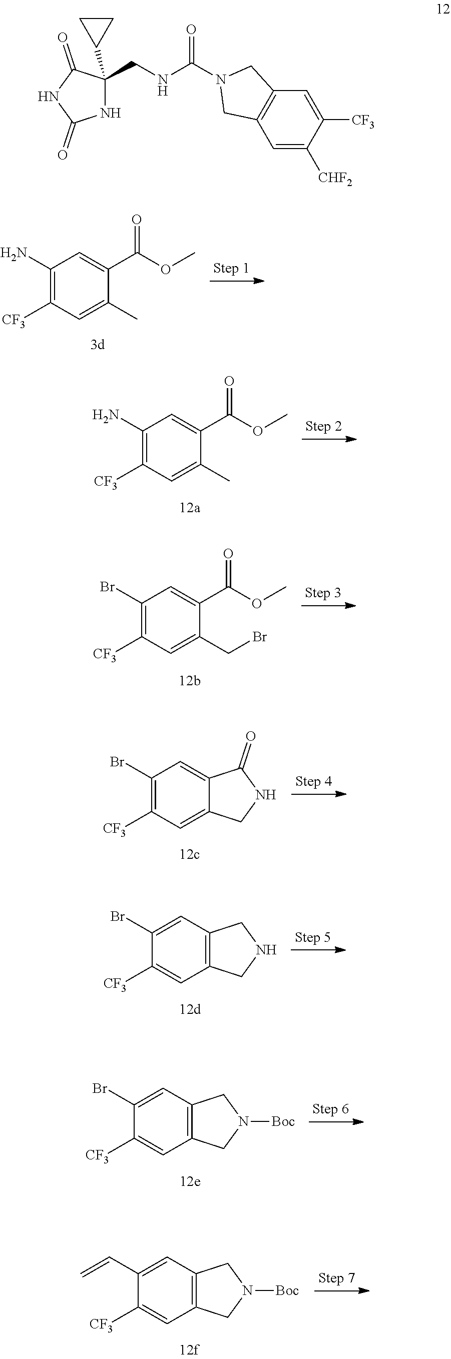

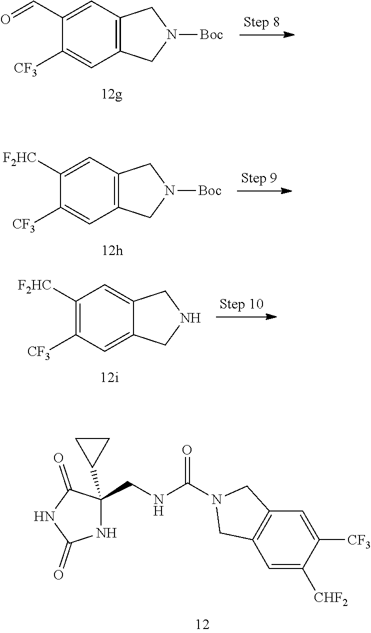 [SMILES: CC(C)(C)OC(=O)N1Cc2cc(C=O)c(C(F)(F)F)cc2C1 ; CC(C)(C)OC(=O)N1Cc2cc(C(F)F)c(C(F)(F)F)cc2C1 ; FC(F)c1cc2c(cc1C(F)(F)F)CNC2 ; O=C1NC(=O)[C@](CNC(=O)N2Cc3cc(C(F)F)c(C(F)(F)F)cc3C2)(C2CC2)N1]

### Step 1

### Methyl 5˜bromo-2-methyl-4-(trifluoromethyl)benzoate 12a

To a suspension of 3d (500 mg, 2.14 mmol) in CH₃CN (20 mL) was added isoamyl nitrite (377 mg, 3.22 mmol) and CuBr₂ (960 mg, 4.30 mmol). After the mixture was stirred at 70° C. overnight, the mixture was cooled down to room temperature and poured into ice water (20 mL). Then the mixture was extracted with EtOAc (50 mL). The organic phase was dried over Na₂SO₄, filtered. The filtrate was concentrated to afford crude 12a (600 mg, 2.02 mmol, 94.20% yield).

¹H NMR (400 MHz, CDCl₃): δ 8.20 (s, 1H), 7.56 (s, 1H), 3.93 (s, 3H), 2.59 (s, 3H).

### Step 2

### Methyl 5-bromo-2-(bromomethyl)-4-(trifluoromethyl)benzoate 12b

To a solution of 12a (100 mg, 336.62 umol) in CCl₄ (3 mL) was added AIBN (2 mg, 10.10 umol) and NBS (72 mg, 403.95 umol), and the mixture was stirred at 70° C. overnight. The mixture was cooled to RT and filtered, the cake was washed with DCM, the filtrate was concentrated in vacuo to give crude 12b which is used directly to next step without further purification.

### Step 3

### 6-bromo-5-(trifluoromethyl)isoindolin-1-one 12c

To a solution of 12b (150 mg, 398.97 umol) in MeOH (1 mL) was added NH₃/MeOH (4 mL), the mixture was stirred at RT overnight. The reaction mixture was concentrated in vacuo, and the residue was purified by silica gel chromatography (EtOAc/hexane=1/5) to give 12c (60 mg, 214.25 umol, 53.70% yield).

¹H NMR (400 MHz, DMSO-d₆): δ 9.03 (brs, 1H), 8.16 (s, 1H), 8.08 (s, 1H), 4.44 (s, 2H).

LCMS: MS m/z (ESI): 280.3 [M+H]⁺.

### Step 4

### 5-bromo-6-(trifluoromethyl)isoindolidine 12d

To a solution of 12c (60 mg, 214.25 umol) in THF (2 mL) was added BH₃/THF (30 mg, 2.14 mmol, 5 mL), and the mixture was heated to 60° C. and stirred overnight. The reaction was quenched with MeOH (5 mL) and the mixture was adjusted pH to 1-2 with 6 M HCl. The mixture was heated to 80° C. and stirred for 1 h. The reaction was cooled to RT and adjusted pH to 7-8 with 6 M NaOH. The product was extracted with EtOAc (60 mL) and the organic phase was dried over anhydrous Na₂SO₄ then concentrated in vacuo. The residue was purified by silica gel chromatography (MeOH/DCM=1/20) to afford 12d (20 mg, 75.17 umol, 35.09% yield).

LCMS; MS m/z (ESI): 266.2 [M+H]⁺.

### Step 5

### Tert-butyl 5-bromo-6-(trifluoromethyl)isoindoline-2-carboxylate 12c

To a solution of 12d (700 mg, 2.63 mmol) in THF (15 mL) was added TEA (1.5 mL) and (Boc)₂O (689 mg, 3.16 mmol), and the mixture was stirred at RT for 3 h. The reaction mixture was concentrated in vacuum and the residue was purified by silica gel chromatography (ethyl acetate/petroleum ether=1/20) to afford 12e (750 mg, 2.05 mmol, 77.85% yield).

LCMS; MS mix (ESI): 310.2 [M+H-56]⁺.

### Step 6

### Tert-butyl 5-(trifluoromethyl)-6-vinylisoindoline-2-carboxylate 12f

To a solution of 12e (750 mg, 2.05 mmol) in 1,4-dioxane (30 mL) was added potassium vinyltrifluoroborate (302 mg, 2.25 mmol), Pd(dppf)Cl₂ (167 mg, 204.82 umol) and K₂CO₃ (849 mg, 6.14 mmol). The reaction mixture was purged with N₂ for three times. The mixture was stirred at 90° C. overnight. Water was added and the mixture was extracted with EtOAc. The combined organic layers were washed with water and brine, dried over anhydrous Na₂SO₄, filtered and concentrated in vacuum. The residue was purified by prep-HPLC using CH₃CN/H₂O as eluent to give 12f (500 mg, 1.60 mmol, 77.91% yield).

¹H NMR (400 MHz, DMSO-d₆): δ 7.81 (d, 1H), 7.71 (d, 1H), 7.02-6.94 (m, 1H), 5.94-5.87 (m, 1H), 5.50 (d, 1H), 4.66-4.58 (m, 4H), 1.47 (s, 9H).

LCMS: MS m/z (ESI): 258.4 [M+H-56]⁺.

### Step 7

### Tert-butyl 5-formyl-6-(trifluoromethyl)isoindoline-2-carboxylate 12g

To a solution of 12f (200 mg, 638.34 umol) in 1,4-dioxane (2 mL) was added NaIO₄ (273 mg, 1.28 mmol) and H₂O (1 mL), and the mixture was stirred at RT before OsO₄ (17 mg, 63.83 umol) was added. The reaction mixture was stirred at RT for 3 h. Saturated sodium bicarbonate aqueous solution was added, and then the reaction mixture was extracted with EtOAc. The combined organic layers were washed with water and brine, dried over anhydrous Na₂SO₄, filtered and concentrated in vacuum. The residue was purified by silica gel chromatography (EtOAc:hexane=1:20) to give 12g (170 mg, 539.19 umol, 84.47% yield).

LCMS: MS m/z (ESI): 260.4 [M+H-56]⁺.

### Step 8

### Tert-butyl 5-(difluoromethyl)-6-(trifluromethyl)isoindoline-2-carboxylate 12h

To a solution of 12g (170 mg, 539.19 umol) in DCM (5 mL) was added EtOH (2.48 mg, 53.92 umol), and the DAST (435 mg, 2.70 mmol) was added dropwise at RT. The reaction was stirred at RT for 3 h. Water was added, and the reaction mixture was extracted with DCM. The combined organic layers were washed with water and brine, dried over anhydrous Na₂SO₄, filtered and concentrated in vacuum. The residue was purified by silica gel column chromatography eluting with ethyl acetate/petroleum ether (1:20) to afford 12h (170 mg, 504.03 umol, 93.5% yield).

LCMS: MS m/z (ESI): 338.3 [M+H]⁺.

### Step 9

### 5-(difluoromethyl)-6-(trifluoromethyl)isoindoline 12i

The solution of 12h (170 mg, 504.03 umol) in HCl/1,4-dioxane (5 mL, 4N) was stirred at RT for 2 h. The reaction mixture was concentrated in vacuum to give 12i (85 mg, 358.39 umol, 71.1% yield).

LCMS: MS m/z (ESI): 238.1 [M+H]⁺.

### Step 10

### (R)—N-((4-cyclopropyl-2,5-dioxoimidazolidin-4-yl)methyl)-5-(difluoromethyl)-6-(trifluoromethyl)isoindoline-2-carboxamide 12

The solution of (4-nitrophenyl) chloroformate (56 mg, 274.85 umol) in DCM (2 mL) was cooled to 0° C. and stirred 20 min. Then a mixture of Int-1B (31 mg, 183.24 umol) and DIEA (71 mg, 549.7 umol, 90.85 uL) in DCM (2 mL) was added dropwise and controlled temperature at 0° C. Then the reaction was stirred at RT overnight. A mixture of 12i (44 mg, 183.24 umol) and TEA (0.2 mL) in DMSO (2 mL) was added and the mixture was heated to 50° C. for 4 h. Water was added, and the reaction mixture was extracted with DCM. The combined organic layers were washed with water and brine, dried over anhydrous Na₂SO₄, filtered and concentrated in vacuum. The crude was purified by prep-HPLC using CH₃CN/H₂O as eluent to give 12 (28 mg, 64.76 umol, 35.3% yield).

¹H NMR (400 MHz, DMSO-d₆) δ 10.48 (s, 1H), 7.92 (s, 1H), 7.90 (s, 1H), 7.46 (s, 1H), 7.28 (t, 1H), 6.40 (t, 1H), 4.72-4.67 (m, 411), 3.58-3.39 (m, 2H), 1.13-1.08 (m, 1H), 0.45-0.39 (m, 2H), 0.35-0.29 (m, 1H), 0.13-0.08 (m, 1H).

¹⁹F NMR (376.5 MHz, DMSO-d₆) δ −56.66-109.77.

LCMS: MS m/z (ESI): 433.1 [M+H]⁺.

The following compounds can be prepared using the similar methods as Examples 1-5

| Example | |
|---|---|
| number | Structure |
| 6 | 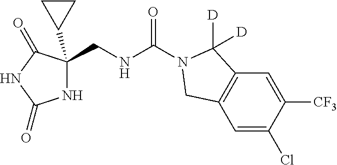 [SMILES: [2H]C1([2H])c2cc(C(F)(F)F)c(Cl)cc2CN1C(=O)NC[C@@]1(C2CC2)NC(=O)NC1=O] |
| 7 | 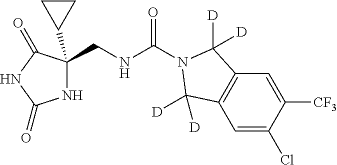 [SMILES: [2H]C1([2H])c2cc(Cl)c(C(F)(F)F)cc2C([2H])([2H])N1C(=O)NC[C@@]1(C2CC2)NC(=O)NC1=O] |
| 10 | 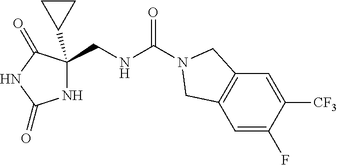 [SMILES: O=C1NC(=O)[C@](CNC(=O)N2Cc3cc(F)c(C(F)(F)F)cc3C2)(C2CC2)N1] |
| 13 | 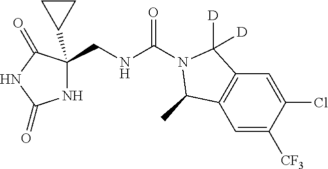 [SMILES: [2H]C1([2H])c2cc(Cl)c(C(F)(F)F)cc2[C@@H](C)N1C(=O)NC[C@@]1(C2CC2)NC(=O)NC1=O] |
| 14 | 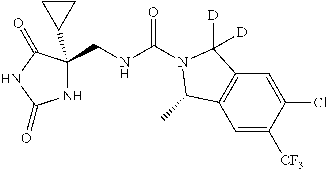 [SMILES: [2H]C1([2H])c2cc(Cl)c(C(F)(F)F)cc2[C@H](C)N1C(=O)NC[C@@]1(C2CC2)NC(=O)NC1=O] |

### Biological Assays

The present disclosure will be further described with reference to the following test examples, but the examples should not be considered as limiting the scope of the disclosure.

### Test Example 1. In Vitro Fluorescence Assay of ADAMTS-4 or ADAMTS-5 Activity

A FRET (fluorescence resonance energy transfer) peptide was cleaved by recombinant ADAMTS-4 or ADAMTS-5 proteins into two separate fragments resulting in an increase of fluorescence signal which was quantified. The peptide was 5-FAM-TEGEARGSVILLK (5-TAMRA)K—NH2, customized from ANASPEC. ADAMTS-4 recombinant protein (4307-AD) and ADAMTS-5 recombinant protein (2198-AD) were purchased from R&D Systems.

An assay buffer containing 50 mM HEPES pH 7.5, 100 mM NaCl, 5 mM CaCl2, 0.1% CHAPS and 5% Glycerol was prepared. A volume of 2.5 μl of compound in the assay buffer was dispensed to a 384-well plate, and 2.5 μl of ADAMTS-4 or ADAMTS-5 protein (final concentration in the reaction was 10 nM) was added. The compounds and proteins were pre-incubated at room temperature for 15 minutes. Then, 5 μl of substrate was added to each well. The final substrate concentrations for ADAMTS-4 and ADAMTS-5 were 15 μM and 8 μM, respectively. The fluorescence signal in each well was determined, after incubation at 37° C. for 3 hours, on a TECAN plate reader (Excitation, 490 nm; Emission, 520 nm).

### Data Analysis:

The data was inputted into GraphPad Prism, and the IC₅₀ values were calculated using function “log (inhibitor) vs. response-Variable slope (four parameters)”, (See Table 1)

TABLE 1
The IC₅₀ values of the exemplified
compounds in the FRET-peptide enzymatic assay.

| Example No. | ADAMTS-5 (IC₅₀, nM) | ADAMTS-4 (IC₅₀, nM) |
|---|---|---|
| 1 | 38 | 31 |
| 1-2 | 23 | 16 |
| 2 | 36 | 42 |
| 2-1 | 9 | 34 |
| 3 | 40 | 39 |
| 4 | 100 | 280 |
| 8 | 22 | 33 |
| 8-1 | 8 | 12 |
| 9 | 30 | 53 |
| 11 | 30 | 92 |

Conclusion: The compounds of the present disclosure have a significant inhibition effect on the enzymatic activities of ADAMTS-4 and ADAMTS-5.

### Test Example 2. Aggrecan-IGD Enzymatic Assay for ADAMTS-5 Inhibitors

In this assay, the enzymatic activity of recombinant ADAMTS-5 protein (2198-AD, R&D Systems) was assayed with a protein substrate, the aggrecan IGD protein. The aggrecan IGD protein is a polypeptide connecting human aggrecan globular domains 1 and 2 (T331-G458) expressed in E. coli with a C-terminal His-tag (BIOTEZ, 30411000). The enzymatic product ARGSVIL-peptide was detected using an ELISA kit from BioTEZ (30510111). An assay buffer containing 50 mM HEPES pH 7.5, 100 mM NaCl, 5 mM CaCl2, 0.1% CHAPS and 5% Glycerol was prepared. Recombinant ADAMTS-5 protein was diluted to 0.3 nM in the assay buffer. Ten μL of buffer and 10 μl of compound solution was transferred to each well of a 96-well plate and incubated at room temperature for 15 minutes. Substrate aggrecan-IGD was diluted to 100 nM with the assay buffer and 20 μl was added to each well. The plate was incubated at 37° C. for 45 minutes. After incubation, the newly generated epitope ARGSVIL-peptides were measured using the Aggrecanase Activity ELISA Assay Kit following the manufacturer's instructions. Then, 100 μl of stop solution was added and the absorbance of each well was read at 450 nM, using 620 nM as reference on a TECAN plate reader.

### Data Analysis:

A standard curve of the ELISA assay was generated in GraphPad Prism using Sigmoidal 4PL function and the corresponding peptide concentrations were calculated based on the standard curve. The IC₅₀ values were calculated using function “log (inhibitor) vs. response-Variable slope (four parameters)”. (See Table 2).

TABLE 2
The IC₅₀ values of the exemplified compounds from
the Aggrecan-IG enzymatic assay.

| Example No. | ADAMTS-5 (IC₅₀, nM) |
|---|---|
| 1 | 60 |
| 1-2 | 26 |
| 2 | 44 |
| 2-1 | 17 |
| 3 | 40 |
| 4 | 140 |
| 8 | 34 |
| 8-1 | 17 |
| 9 | 34 |

Conclusion: The compounds of the present disclosure have a significant inhibition effect on the enzymatic activity of ADAMTS-5.

### Test Example 3. Mouse Cartilage Explant Assay

In this assay, fresh mouse femoral head cartilage was treated with IL-1a protein (Sigma-Aldrich, 12778) in culture media, which induced the cartilage catabolism. Then, the GAGs attached to the cleaved aggrecan fragments (released in the media) and the GAGs attached to the intact aggrecan were measured by dimethylmethylene blue dye in the Glycosaminoglycans Assay Kit (Chondrex, 6022).

Femoral head cartilage samples were isolated from mice (25 days old, male, C57BL/6, from Charles River Lab) and put into 2.0 mL tubes filled-up with media (DMEM, 10% FBS, 4 mM Glutamine, penicillin-streptomycin, 20 mM HEPES). 200 μl of media without FBS was added to each well of a 48-well plate, and one piece of cartilage was transferred to a well in the plate. Then the media was aspirated, and compounds and IL-1α protein were added to the plate in a total volume of 400 μl of fresh media without FBS. The final concentration of IL-1α was 1 ng/mL. The plate was incubated at 37° C. for 72 hours in a humidified incubator with 5% CO₂ supply.

The supernatant was transferred to a 1.5 mL tube and kept at −20° C. Each cartilage sample was transferred to another 1.5 mL tube containing 400 μl of freshly made papain solution. The papain solution contained 125 μg/mL papain (Sigma-Aldrich, P3125), 0.1 M sodium acetate (Sigma-Aldrich. S7899), pH 5.5 and 5 mM EDTA and 5 mM L-cysteine-HCl (Sigma-Aldrich, C7880). The cartilage samples were kept rocking in a 60° C. water bath for 24 hours.

The lysates were vortexed for 10 seconds and spinned at 10,000 rpm for 2 minutes. Both the supernatant and the lysate samples were diluted with PBS and mixed with 100 μL of dye from the Glycosaminoglycans Assay Kit. The optical density from each well was determined with a TECAN plate reader set to a wavelength of 525 nm,

### Data Analysis:

The concentrations of GAGs in the supernatant and lysates were determined based on the standard curve with a dose range of chondroitin sulfate provided in the kit. The percentage of GAG release was calculated as the following:

$$GAG \% = ( \frac{[ GAG ] supernatant}{[ GAG ] supernatant + [ GAG ] lysate} ) \times 100 \% .$$

The test compound effect was expressed as the percent of inhibition using the following formula:

$$Inhibition \% = ( 1 - \frac{GAG \% ( Compound + IL 1 α ) - GAG \% ( Vehicle )}{GAG \% ( Vehicle + IL 1 α ) - GAG \% ( Vehicle )} \times 100 \% .$$

The inhibition data of selected exemplified compounds at 2 μM and 20 μM concentrations were listed in Table 3.

TABLE 3
The IC₅₀ values of the exemplified
compounds from the mouse explant assay.

| Example No. | Inhibiton % at 2 μM | Inhibition % at 20 μM |
|---|---|---|
| 1-2 | 47 | 87 |
| 2-1 | 14 | 86 |
| 4 | 0 | 94 |
| 8-1 | 61 | 95 |

The foregoing embodiments and examples are provided for illustration only and are not intended to limit the scope of the disclosure. Various changes and modifications to the disclosed embodiments will be apparent to those skilled in the art based on the present disclosure, and such changes and modifications may be made without departure from the spirit and scope of the present disclosure. All literature cited are incorporated herein by reference in their entireties without admission of them as prior art.

What is claimed is:

1. A compound of formula (I), or a tautomer, mesomer, racemate, enantiomer, diastereomer, or mixture thereof, or a pharmaceutically acceptable salt, solvate or prodrug thereof: 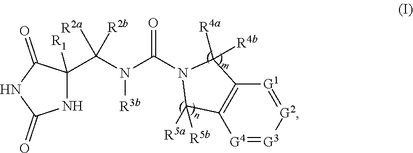
wherein:
G¹, G², G³ and G⁴ are each independently N or CR⁶, provided that no more than two of them are N;
R¹ is selected from the group consisting of hydrogen, alkyl, haloalkyl, hydroxyalkyl, cycloalkyl, heterocyclyl and heteroaryl, wherein the alkyl, cycloalkyl, heterocyclyl or heteroaryl is optionally substituted with one or more groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NHC(═O)R^{11a}, NHC(═O)OR^{11a}, cycloalkyl, heterocyclyl, and heteroaryl;
R^{2a} and R^{2b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;
R^{3b} is selected from the group consisting of hydrogen, alkyl, hydroxy, haloalkyl, hydroxyalkyl, amino, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;
or alternatively two of R^{2a}, R^{2b} and R^{3b} together form cycloalkyl or heterocyclyl;
R^{4a}, R^{4b}, R^{5a} and R^{5b} are each identical or different, and each is independently selected from the group consisting of hydrogen, deuterium, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, cycloalkyl, heterocyclyl, and heteroaryl;
or alternatively two of R^{4a}, R^{4b}, R^{5a} and R^{5b} together form cycloalkyl or heterocyclyl;
each R⁶ is identical or different and at each occurrence is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
each of R^{11a}, R^{12a}, R^{13a}, and R^{14a} is independently selected from the group consisting of hydrogen, alkyl, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, hydroxy, alkoxy, alkyl, and cycloalkyl;
each of R^{11b}, R^{12b}, R^{13b}, and R^{14b} is independently selected from the group consisting of hydrogen and alkyl, wherein alkyl is optionally substituted with one or more groups independently selected from the group consisting of halogen, hydroxyl and alkoxy;
n is 1 or 2; and
m is 1 or 2.

2. The compound of claim 1, being a compound of formula (II), or a tautomer, mesomer, racemate, enantiomer, diastereomer, or mixture thereof, or a pharmaceutically acceptable salt, solvate or prodrug thereof: 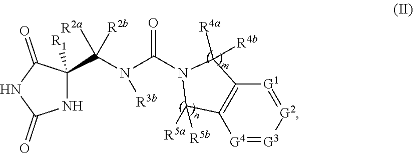
wherein G¹, G², G³, G⁴, R¹, R^{2a}, R^{4a} to R^{5a}, R^{2b} to R^{5b}, n and m are each as defined in claim 1.

3. The compound of claim 1, or a tautomer, mesomer, racemate, enantiomer, diastereomer, or mixture thereof, or a pharmaceutically acceptable salt, solvate or prodrug thereof, wherein G¹ and G² are each independently N or CR⁶; G³ and G⁴ are each CR⁶; and R⁶ is as defined in claim 1.

4. The compound according to claim 1, being a compound of formula (III) or (IIIa), or a tautomer, mesomer, racemate, enantiomer, diastereomer, or mixture thereof, or a pharmaceutically acceptable salt, solvate or prodrug thereof: 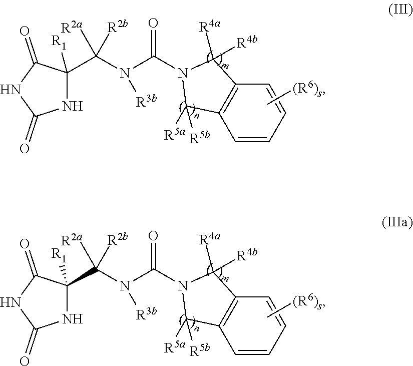
wherein s is 0, 1, 2, 3 or 4; and
R¹, R^{2a}, R^{4a} to R^{5a}, R^{2b} to R^{5b}, R⁶, n and m are each as defined in claim 1.

5. The compound of according to claim 1, or a tautomer, mesomer, racemate, enantiomer, diastereomer, or mixture thereof, or a pharmaceutically acceptable salt, solvate or prodrug thereof, wherein R¹ is cyclopropyl.

6. The compound according to claim 1, or a tautomer, mesomer, racemate, enantiomer, diastereomer, or mixture thereof, or a pharmaceutically acceptable salt, solvate or prodrug thereof, wherein R^{2a} and R^{2b} is hydrogen.

7. The compound according to claim 1, being a compound of formula (IV) or (IVa), or a tautomer, mesomer, racemate, enantiomer, diastereomer, or mixture thereof, or a pharmaceutically acceptable salt, solvate or prodrug thereof: 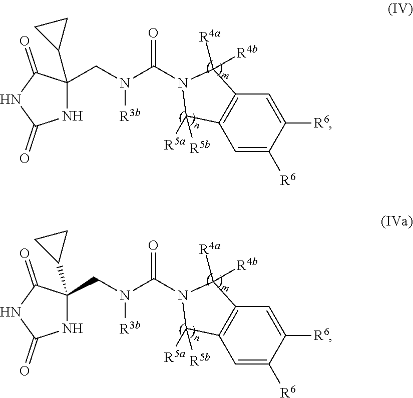
wherein:
R^{4a}, R^{5a}, R^{3b} to R^{5b}, R⁶, n and m are each as defined in claim 1.

8. The compound according to claim 1, or a tautomer, mesomer, racemate, enantiomer, diastereomer, or mixture thereof, or a pharmaceutically acceptable salt, solvate or prodrug thereof, wherein R^{3b} is selected from the group consisting of hydrogen and alkyl.

9. The compound according to claim 1, or a tautomer, mesomer, racemate, enantiomer, diastereomer, or mixture thereof, or a pharmaceutically acceptable salt, solvate or prodrug thereof, wherein R^{4a}, R^{4b}, R^{5a} and R^{5b} are each identical or different, and each is independently selected from the group consisting of hydrogen, deuterium and alkyl.

10. The compound according to claim 1, or a tautomer, mesomer, racemate, enantiomer, diastereomer, or mixture thereof, or a pharmaceutically acceptable salt, solvate or prodrug thereof, wherein each R⁶ is identical or different and at each occurrence is independently selected from the group consisting of hydrogen, halogen and haloalkyl.

11. The compound of according to claim 1, or a tautomer, mesomer, racemate, enantiomer, diastereomer, or mixture thereof, or a pharmaceutically acceptable salt, solvate or prodrug thereof, wherein 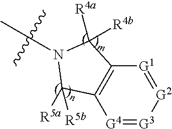
is selected from the group consisting of 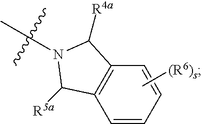
R^{4a}, R^{5a} and R⁶ are as defined in claim 1; and s is 1 or 2.

12. The compound according to claim 1, or a tautomer, mesomer, racemate, enantiomer, diastereomer, or mixture thereof, or a pharmaceutically acceptable salt, solvate or prodrug thereof, wherein the compound is selected from the group consisting of:

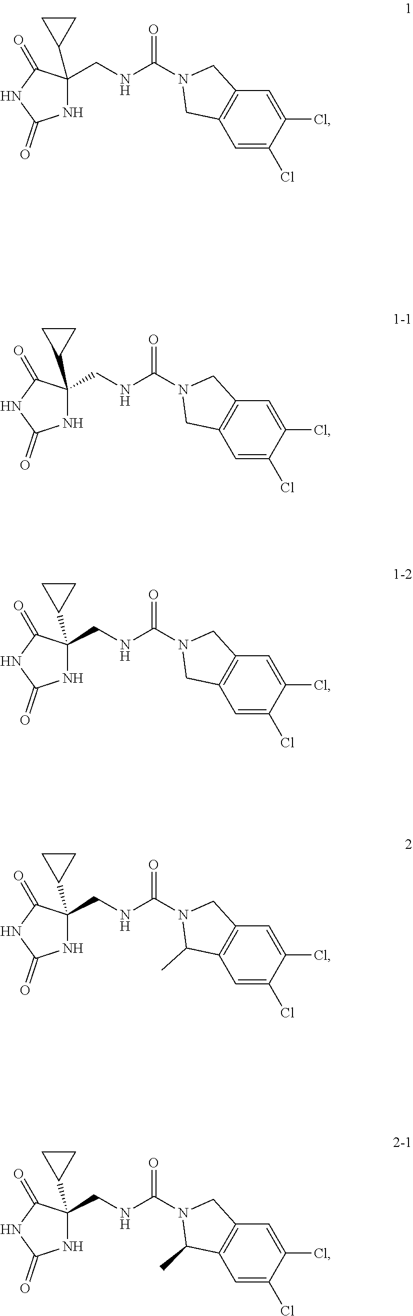 [SMILES: O=C1NC(=O)C(CNC(=O)N2Cc3cc(Cl)c(Cl)cc3C2)(C2CC2)N1 ; O=C1NC(=O)[C@@](CNC(=O)N2Cc3cc(Cl)c(Cl)cc3C2)(C2CC2)N1 ; O=C1NC(=O)[C@](CNC(=O)N2Cc3cc(Cl)c(Cl)cc3C2)(C2CC2)N1 ; CC1c2cc(Cl)c(Cl)cc2CN1C(=O)NC[C@@]1(C2CC2)NC(=O)NC1=O ; C[C@@H]1c2cc(Cl)c(Cl)cc2CN1C(=O)NC[C@@]1(C2CC2)NC(=O)NC1=O]

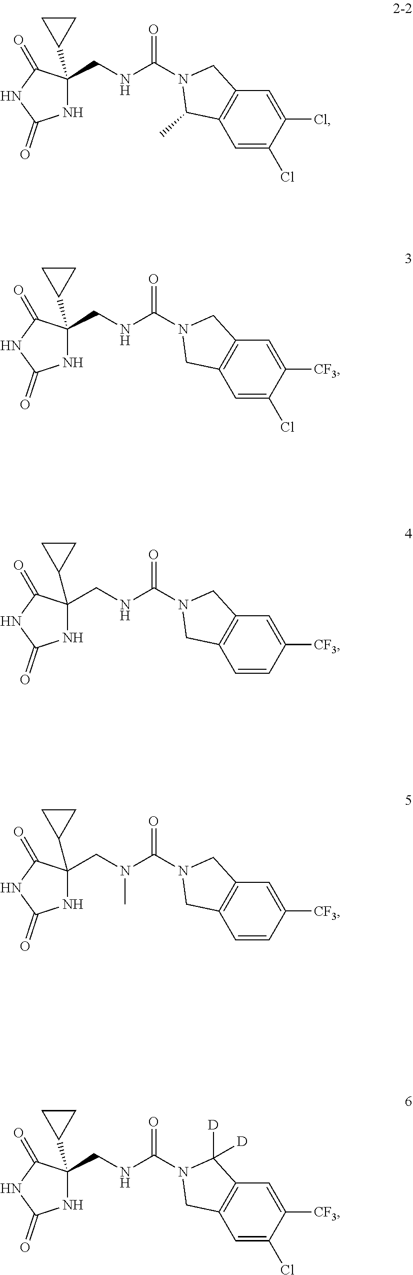 [SMILES: C[C@H]1c2cc(Cl)c(Cl)cc2CN1C(=O)NC[C@@]1(C2CC2)NC(=O)NC1=O ; O=C1NC(=O)[C@](CNC(=O)N2Cc3cc(Cl)c(C(F)(F)F)cc3C2)(C2CC2)N1 ; O=C1NC(=O)C(CNC(=O)N2Cc3ccc(C(F)(F)F)cc3C2)(C2CC2)N1 ; CN(CC1(C2CC2)NC(=O)NC1=O)C(=O)N1Cc2ccc(C(F)(F)F)cc2C1 ; [2H]C1([2H])c2cc(C(F)(F)F)c(Cl)cc2CN1C(=O)NC[C@@]1(C2CC2)NC(=O)NC1=O]

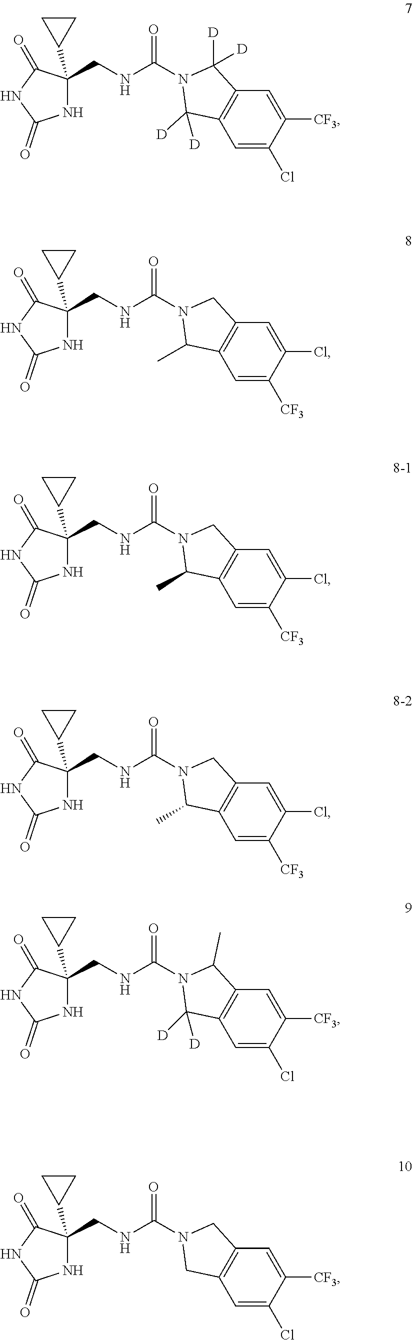 [SMILES: [2H]C1([2H])c2cc(Cl)c(C(F)(F)F)cc2C([2H])([2H])N1C(=O)NC[C@@]1(C2CC2)NC(=O)NC1=O ; CC1c2cc(C(F)(F)F)c(Cl)cc2CN1C(=O)NC[C@@]1(C2CC2)NC(=O)NC1=O ; C[C@@H]1c2cc(C(F)(F)F)c(Cl)cc2CN1C(=O)NC[C@@]1(C2CC2)NC(=O)NC1=O ; C[C@H]1c2cc(C(F)(F)F)c(Cl)cc2CN1C(=O)NC[C@@]1(C2CC2)NC(=O)NC1=O ; [2H]C1([2H])c2cc(Cl)c(C(F)(F)F)cc2C(C)N1C(=O)NC[C@@]1(C2CC2)NC(=O)NC1=O ; O=C1NC(=O)[C@](CNC(=O)N2Cc3cc(Cl)c(C(F)(F)F)cc3C2)(C2CC2)N1]

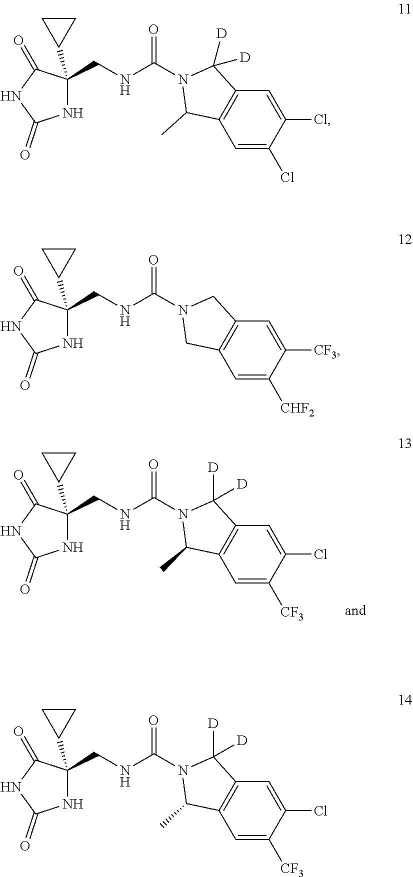 [SMILES: [2H]C1([2H])c2cc(Cl)c(Cl)cc2C(C)N1C(=O)NC[C@@]1(C2CC2)NC(=O)NC1=O ; O=C1NC(=O)[C@](CNC(=O)N2Cc3cc(C(F)F)c(C(F)(F)F)cc3C2)(C2CC2)N1 ; [2H]C1([2H])c2cc(Cl)c(C(F)(F)F)cc2[C@@H](C)N1C(=O)NC[C@@]1(C2CC2)NC(=O)NC1=O ; [2H]C1([2H])c2cc(Cl)c(C(F)(F)F)cc2[C@H](C)N1C(=O)NC[C@@]1(C2CC2)NC(=O)NC1=O]

13. A process of preparing the compound of formula (I) according to claim 1, or a tautomer, mesomer, racemate, enantiomer, diastereomer, or mixture thereof, or a pharmaceutically acceptable salt, solvate or prodrug thereof comprising a step of: 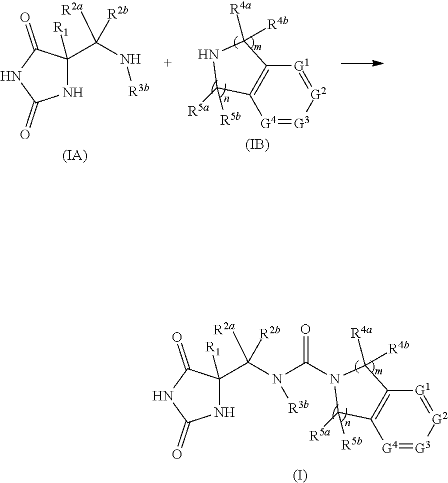
reacting a compound of formula (IA) with a compound of formula (IB) in the presence of an activating reagent to obtain the compound of formula (I) or a tautomer, mesomer, racemate, enantiomer, diastereomer, or mixture thereof, or a pharmaceutically acceptable salt, solvate or prodrug thereof, wherein:
G¹, G², G³, G⁴, R¹, R^{2a}, R^{4a} to R^{5a}, R^{2b} to R^{5b}, n and m are each as defined in claim 1.

14. A pharmaceutical composition, comprising a compound according to claim 1, or a tautomer, mesomer, racemate, enantiomer, diastereomer, or mixture thereof, or a pharmaceutically acceptable salt, solvate or prodrug thereof, and a pharmaceutically acceptable carrier.

15. A method of inhibiting ADAMTS-5 and/or ADAMTS-4, comprising administering to a subject in need thereof a therapeutically effective amount of a compound according to claim 1, or a tautomer, mesomer, racemate, enantiomer, diastereomer, or mixture thereof, or a pharmaceutically acceptable salt, solvate, prodrug, or pharmaceutical composition thereof.

16. A method of preventing and/or treating an inflammatory condition, a disease involving degradation of cartilage and/or disruption of cartilage homeostasis, comprising administering to a subject in need thereof a therapeutically effective amount of a compound according to claim 1, or a tautomer, mesomer, racemate, enantiomer, diastereomer, or mixture thereof, or a pharmaceutically acceptable salt, solvate, prodrug, or pharmaceutical composition thereof.

17. A method of preventing and/or treating arthritis, comprising a step of administering to a subject in need thereof a therapeutically effective amount of a compound according to claim 1, or a tautomer, mesomer, racemate, enantiomer, diastereomer, or mixture thereof, or a pharmaceutically acceptable salt, solvate, prodrug, or pharmaceutical composition thereof.

18. The method of claim 17, wherein the arthritis is selected from the group consisting of rheumatoid arthritis, psoriatic arthritis, osteoarthrosis and hypertrophic arthritis.
